(19) United States
(12) Patent Application Publication
(10) Pub. No.: US 2026/0250643 A1
(43) Pub. Date: Aug. 27, 2026

(54) HSV GENE EXPRESSION VECTOR AND BXB1 INTEGRASE-MEDIATED RECOMBINATION SYSTEM FOR HIGH-THROUGHPUT CLONING

(71) Applicant: Albert Einstein College of Medicine, Bronx, NY (US)

(72) Inventors: WIlliam R. Jacobs, Jr., Pelham, NY (US); Anna De Oliveira, Boston, MA (US); Katherine Kaugars, Boston, MA (US); Regy Lukose, Flanders, NJ (US)

(21) Appl. No.: 18/998,547

(22) PCT Filed: Jul. 27, 2023
(86) PCT No.: PCT/US2023/071074

§ 371 Date: Jan. 27, 2025

Related U.S. Application Data
(60) Provisional application No. 63/392,666, filed on Jul. 27, 2022.

(51) Int. Cl. C12N 7/00; A61K 39/00; A61K 39/04; A61K 39/245; A61K 39/39; A61K 39/395; A61K 39/42; A61P 31/22; C07K 14/035; C12N 15/869

(52) U.S. Cl. CPC C12N 7/00; A61K 39/04; A61K 39/245; A61K 39/39; A61K 39/39516; A61K 39/42; A61P 31/22; C07K 14/035; C12N 15/8695; A61K 2039/525; A61K 2039/53; A61K 2039/55; C12N 2710/16611; C12N 2710/16634; C12N 2710/16643; C12N 2830/34

(57) ABSTRACT

A herpes simplex virus (HSV) gene vaccine and expression vector and/or vaccine vector comprising an HSV genome comprising: a complete deletion of glycoprotein G-encoding gene, glycoprotein J-encoding gene, glycoprotein D-encoding gene, and C glycoprotein I-encoding gene; a heterologous nucleic acid comprising an expression cassette and inserted in a region of the genome from which the glycoprotein G-encoding gene, glycoprotein J-encoding gene, glycoprotein D-encoding gene, and glycoprotein I-encoding gene have been deleted; an attL sequence; and an attR sequence, wherein the attL sequence is adjacent to a first end of the expression cassette and the attR sequence is adjacent to a second end of the expression cassette, and wherein the expression cassette comprises in operable communication a promoter and at least one gene encoding a heterologous protein.

### CROSS REFERENCE TO RELATED APPLICATIONS

This application claims priority from Provisional Application No. 63/392,666 filed on Jul. 27, 2022, incorporated in its entirety herein.

### FEDERAL RESEARCH STATEMENT

This invention was made with government support under grant number R01 AI117321 and AI26170 awarded by the National Institute of Health (NIH). The government has certain rights in the invention.

### INCORPORATION-BY-REFERENCE OF ELECTRONICALLY FILED MATERIAL

The Instant Application contains a Sequence Listing which has been submitted electronically in XML format and is hereby incorporated by reference in its entirety. Said XML copy, created on Jul. 26, 2023 is named “XVS0012PCT Sequence Listing xml.XML” and is 81,920 bytes in size. The Sequence Listing does not go beyond the disclosure in the application as filed.

### BACKGROUND

Emerging viruses with the potential for epidemics and pandemics are a constant and tangible threat to national and global health. Robust and adaptable tools are required that can quickly develop novel and effective vaccines. One strategy is to use genetically engineered viruses to create vaccine platforms for delivery of antigens. These viruses would need maximal capacity to efficiently express specific immunogenic proteins from target pathogens.

Herpes simplex virus (HSV), a member of the Alphaherpesvirinae subfamily, has been explored as a vector for oncolytic viral therapy and gene therapy (Goins W. F., et al., Methods Mol Biol., 2020, 2060:73-90; Fukuhara H, et al., Cancer Sci., 2016, 107(10):1373-9). HSV has broad cell tropism, which includes proliferating (e.g. epithelial) and quiescent cells (e.g. neurons), and its large capacity for the insertion of exogenous DNA. However, the use of HSV as a vaccine vector has been limited by the low rates of recombination in commonly used techniques, like homologous recombination. Putative vaccines against HSV are in development, including those derived from live, attenuated HSV strains. The efficacy of a vaccine based on an HSV-2 strain with a deletion of glycoprotein D in the genome (referred to as AgD-2) in the protection against otherwise lethal infection and disease caused by HSV-1 and HSV-2 challenge viruses has been previously demonstrated (Petro C., et al., Elife., 2015, vol. 4; Petro, C. D., et al., JCI Insight. 2016, 1(12)).

Despite progress, additional vaccine platforms are needed that can respond rapidly and affordably to current and emerging pathogenic threats.

The present invention addresses this need for new and improved vaccine platforms.

### SUMMARY OF THE INVENTION

The present disclosure provides an isolated, recombinant herpes simplex virus-2 (HSV-2) having a complete deletion of a glycoprotein G-encoding gene, a glycoprotein J-encoding gene, a glycoprotein D-encoding gene, and a glycoprotein I-encoding gene, in the genome thereof. The recombinant HSV-2 can be used as a vaccine for protection against infection with herpes simplex virus type 1 (HSV-1) or HSV-2 or coinfection with HSV-1 and HSV-2. In one aspect, when the recombinant HSV-2 comprises a recombination sequence inserted in a region of the genome from which the genes for glycoproteins G, J, D, I have been deleted, the recombinant can be used as a vaccine vector for expressing and eliciting an immune response to one or more transgene or heterologous antigen in a subject, in addition to protection against herpes simplex virus infection. In one aspect, the recombination sequence comprises a Bxb1 attB sequence from Mycobacterium smegmatis.

In an aspect, provided herein is a virion of an isolated, recombinant HSV-2 having a complete deletion of a glycoprotein G-encoding gene, a glycoprotein J-encoding gene, a glycoprotein D-encoding gene, and a glycoprotein I-encoding gene in the genome thereof. In one aspect, the virion of the recombinant HSV-2 further comprises a recombination sequence inserted in a region of the genome from which the genes for glycoproteins G, J, D, I have been deleted, wherein the recombination sequence comprises a Bxb1 attB sequence from Mycobacterium smegmatis.

Also provided is a method of producing a virion of a recombinant herpes simplex virus-2 (HSV-2), having a complete deletion of a glycoprotein G-encoding gene, a glycoprotein J-encoding gene, a glycoprotein D-encoding gene, and a glycoprotein I-encoding gene in the genome thereof and comprising a HSV-1 or HSV-2 glycoproteins G, J, D, I on a lipid bilayer thereof, comprising infecting a cell comprising a heterologous nucleic acid encoding a HSV-1 or HSV-2 glycoprotein G, glycoprotein J, glycoprotein D, and glycoprotein I with a recombinant herpes simplex virus-2 (HSV-2) having a deletion of an HSV-2 glycoprotein G-encoding gene, a glycoprotein J-encoding gene, a glycoprotein D-encoding gene, and a glycoprotein I-encoding gene in the genome thereof under conditions permitting replication of the recombinant herpes simplex virus-2 (HSV-2) and recovering a recombinant HSV-2 virion produced by the cells. In an embodiment, the recombinant HSV-2 is Δ(GJDI)-2. In one aspect, the recombinant HSV-2 comprises a recombination sequence inserted in a region of the genome from which the genes for glycoproteins G, J, D, I have been deleted, wherein the recombination sequence comprises a Bxb1 attB sequence from Mycobacterium smegmatis. In one aspect, the recombinant HSV-2 with the recombination sequence is Δ(GJDI)::attB.

Also provided is a recombinant nucleic acid having the same sequence as a genome of a wild-type HSV-2 except that the recombinant nucleic acid does not comprise a sequence encoding a glycoprotein G, a glycoprotein J, a glycoprotein D, and a glycoprotein I. In one aspect, the recombinant HSV-2 comprises a recombination sequence inserted in a region of the genome from which the genes for glycoproteins G, J, D, I have been deleted, wherein the recombination sequence comprises a Bxb1 attB sequence from Mycobacterium smegmatis.

Also provided is an isolated, recombinant herpes simplex virus type 2 (HSV-2), having a complete deletion of a glycoprotein G-encoding gene, a glycoprotein J-encoding gene, a glycoprotein D-encoding gene, and a glycoprotein I-encoding gene and further comprising a heterologous nucleic acid comprising an expression cassette and inserted in a region of the HSV genome from which the glycoprotein G-encoding gene, glycoprotein J-encoding gene, glycoprotein D-encoding gene, and glycoprotein I-encoding gene have been deleted, the expression cassette comprising in operable communication a promoter and at least one gene encoding a heterologous protein or heterologous antigen or transgene, and having an attL sequence adjacent to a first end of the expression cassette and an attR sequence adjacent to a second end of the expression cassette.

The present disclosure also provides a herpes simplex virus (HSV) gene expression vector or vaccine vector for expressing one or more heterologous antigen, heterologous protein, or transgene, comprising an HSV-2 genome comprising: a complete deletion of a glycoprotein G-encoding gene, a glycoprotein J-encoding gene, a glycoprotein D-encoding gene, and a glycoprotein I-encoding gene; a heterologous nucleic acid comprising an expression cassette and inserted in a region of the HSV genome from which the genes encoding glycoproteins G, J, D, and I have been deleted, the expression cassette comprising in operable communication a promoter and at least one gene encoding a heterologous protein or heterologous antigen or transgene, and having an attL sequence adjacent to a first end of the expression cassette and an attR sequence adjacent to a second end of the expression cassette.

Also provided is a method of producing an isolated, recombinant herpes simplex virus (HSV) gene expression vector expressing one or more heterologous protein or heterologous antigen or transgene, the method comprising: providing a genetically modified HSV-2 comprising a genome comprising a complete deletion of glycoprotein G-encoding gene, glycoprotein J-encoding gene, glycoprotein D-encoding gene, and glycoprotein I-encoding gene, and a first recombination sequence inserted in a region of the genome from which the glycoprotein G-encoding gene, glycoprotein J-encoding gene, glycoprotein D-encoding gene, and glycoprotein I-encoding gene have been deleted; providing a heterologous nucleic acid comprising a second recombination sequence and an expression cassette comprising the one or more gene encoding the one or more heterologous protein; contacting the genome and the heterologous nucleic acid in the presence of a bacteriophage Bxb1 serine integrase under conditions in which sequence-specific recombination occurs between the first recombination sequence and the second recombination sequence to provide a recombined HSV genome; and transfecting a complementing cell with the recombined HSV genome to obtain the HSV gene expression vector, wherein the first recombination sequence comprises a Bxb1 attB sequence from Mycobacterium smegmatis and the second recombination sequence comprises an attP sequence from bacteriophage Bxb1.

Also disclosed herein is a recombination system for high-throughput cloning, comprising: a first recombination partner comprising a genetically modified herpes simplex virus having a genome comprising a complete deletion of a glycoprotein G-encoding gene, a glycoprotein J-encoding gene, a glycoprotein D-encoding gene, and a glycoprotein I-encoding gene, and a first recombination sequence; and a second recombination partner comprising a nucleic acid encoding a second recombination sequence, and an expression cassette, wherein the first recombination sequence comprises a Bxb1 attB sequence from Mycobacterium smegmatis and the second recombination sequence comprises an attP sequence from bacteriophage Bxb1, and wherein the expression cassette comprises in operable communication a promoter and at least one gene encoding a heterologous protein.

This disclosure also provides a method of expressing a heterologous protein in a host cell, the method comprising: providing the recombination system disclosed herein; contacting the first recombination partner and the second recombination partner in the presence of a bacteriophage Bxb1 serine integrase and under conditions in which sequence-specific recombination occurs between the first recombination sequence and the second recombination sequence to provide a recombined HSV genome; transfecting a complementing cell with the recombined HSV genome; recovering an HSV virus or virion from the transfected complementing cell; and infecting the host cell with the HSV virus or virion to express the heterologous protein.

An isolated cell is provided comprising therein a recombinant HSV-2 genome as described herein or a recombinant HSV-1 gene as described herein, wherein the cell is not present in a human being.

Also provided is a vaccine composition comprising the recombinant HSV-2 virus as described herein, or the virion as described herein.

A pharmaceutical composition comprising the recombinant HSV-2 virus as described herein, or the virion as described herein, and a pharmaceutically acceptable carrier.

Also provided is a method of eliciting an immune response in a subject comprising administering to the subject an amount of (i) the recombinant HSV-2 virus as described herein; (ii) a virion thereof as described herein, (iii) the vaccine as described herein; (iv) a composition as described herein; or (v) a pharmaceutical composition as described herein, in an amount effective to elicit an immune response in a subject.

Also provided is a method of treating an HSV-1, HSV-2 or HSV-1 and HSV-2 co-infection in a subject or treating a disease caused by an HSV-1, HSV-2 or co-infection in a subject comprising administering to the subject an amount of (i) the recombinant HSV-2 virus as described herein; (ii) a virion thereof as described herein, (iii) the vaccine as described herein; (iv) a composition as described herein; or (v) a pharmaceutical composition as described herein, in an amount effective to treat an HSV-1, HSV-2 or co-infection or treat a disease caused by an HSV-1, HSV-2 or co-infection in a subject.

Also provided is a method of vaccinating a subject for HSV-1, HSV-2 or co-infection comprising administering to the subject an amount of (i) the recombinant HSV-2 virus as described herein; (ii) a virion thereof as described herein, (iii) the vaccine as described herein; (iv) a composition as described herein; or (v) a pharmaceutical composition as described herein, in an amount effective to vaccinate a subject for HSV-1, HSV-2 or co-infection.

Also provided is a method of immunizing a subject against HSV-1, HSV-2 or co-infection comprising administering to the subject an amount of (i) the recombinant HSV-2 virus as described herein; (ii) a virion thereof as described herein, (iii) the vaccine as described herein; (iv) a composition as described herein; or (v) a pharmaceutical composition as described herein, in an amount effective to immunize a subject against HSV-1, HSV-2 or co-infection.

Also provided is a method of eliciting an immune response against a heterologous antigen in a subject comprising administering to the subject an amount of (i) the recombinant HSV-2 virus having an expression cassette comprising a gene encoding the heterologous antigen as described herein; (ii) a virion thereof as described herein, (iii) the vaccine as described herein; (iv) a composition as described herein; or (v) a pharmaceutical composition as described herein, in an amount effective to elicit an immune response in a subject.

In an embodiment of the vaccines, compositions and pharmaceutical compositions, and of the methods of use thereof, the amount of recombinant HSV-2 is an amount of pfu of recombinant HSV-2 effective to achieve the stated aim.

The above described and other features are exemplified by the following figures and detailed description.

### BRIEF DESCRIPTION OF THE DRAWINGS

The following figures are exemplary embodiments wherein the like elements are numbered alike.

FIGS. 1A and B illustrate the construction of Δg(GJDI)-2::attB genome. The HSV-2 strain G was used as the backbone for the construction of the recombinant viral vector. A cosmid, pBRL962, containing the attB sequence flanked by the homologous sequences to US3 and US8 was used for the deletion of the US4 (gG) to US7 (gI) genes and insertion of the attB site by homologous recombination (HR).

FIGS. 2A-C show construction of attP-shuttle plasmids. 2A) Shuttle plasmids were designed as minimal plasmids (pMAE) which contained the Bxb1 attP site upstream the CMV promoter and a multiple cloning site (MCS). 2B, 2C) Reporter genes encoding for green fluorescent protein (GFP), pMAE1, or combination of blue fluorescent protein (BFP), GFP, and red fluorecsent protein mCherry, BFP-GFP-mCherry, pMAE2, were inserted into the MCS of pMAE.

FIGS. 3A and B is a schematic showing production of Δg(GJDI)-2::GFP recombinant using Bxb1 integrase-mediated recombination into the viral genome.

FIGS. 4A and B show analysis of integrase-mediated recombination. 4A) genome of Δg(GJDI)-2::GFP showing location of forward and reverse primers. 4B) Bxb1 integrase-mediated recombination into viral genome Δg(GJDI)-2::attB, Δg(GJDI)-2::GFP, and Δg(GJDI)-2::BFP-GFP-mCherry was analyzed by PCR using primers flanking the insertion region in the Δg(GJDI)-2 viral genome, and the expected PCR amplicon size for each of the recombinants.

FIGS. 5A-E show production and expression of inserted antigen. The Mycobacterium tuberculosis(Mtb) genes, dnaK and groEL-2, were inserted into pMAE90 (5A) shuttle plasmid, to produce pMAE::dnaK (5B) and pMAE::groEL-2 (5C), as described in the Examples below. 5D, 5E) Vero cells were transfected with either the pMAE::dnaK or pMAE::groEL-2 recombinants and expression of the respective Mtb proteins was analysed by immunofluorescence.

FIGS. 6A-F demonstrate that Δg(GJDI)-2 is fully protective against HSV-2 and HSV-1 challenge. Mice were primed and boosted with 5×10⁶ PFU of Δg(GJDI)-2, or 5×10⁶ PFU of ΔgD-2::RFP, and mock immunized with lysate from the complementing cell line (VD60 lysate). Mice were then challenged with either 100×LD₅₀ of HSV-1 or 10×LD₉₀ of HSV-2 intradermally. Mice were monitored for 2 weeks for survival (6A, 6B), neurological disease (6C, 6D), and epithelial disease (6E, 6F). Survival was analyzed using a Gehan-Breslow-Wilcoxon test and the epithelial and neurological disease scoring was analyzed with two-way ANOVA, main column effect (****p<0.0001) and represented as mean±SD; n=10 mice/group.

FIGS. 7A-C show results of serum analysis of Δg(GJDI)-2-vaccinated mice, compared with ΔgD-2::RFP-vaccinated mice. Mice were vaccinated with 5×10⁶ PFU Δg(GJDI)-2, 5×10⁶ PFU ΔgD-2::RFP, or VD60 cell lysate and then boosted with the same vaccination 3 weeks later. At week 6, sera were obtained from the mice. The sera were tested with an anti-HSV-2 ELISA for total IgG (7A) and IgG2c (7B) antibody titers. In addition, sera were also analyzed using an FcγRIV activation assay using HSV-2-infected Vero cells as targets (7C). Antibody levels and fold FcγRIV activation were analyzed using one-way ANOVA (**p<0.01, ***p<0.001, ****p<0.0001) and represented as mean±SD; n=10 mice/group in A and B and n=4 mice per group in C.

FIGS. 8A-C. Serum analysis of Δg(GJDI)-2-vaccinated mice, compared with ΔgD-2::RFP-vaccinated mice. Mice were vaccinated with 5×10⁶ PFU Δg(GJDI)-2, 5×10⁶ PFU ΔgD-2::RFP, or VD60 cell lysate and then boosted with the same vaccination three weeks later. At week 6, sera were obtained from the mice. The sera were tested with ELISA for IgG1 (8A), IgG2b (8B), and IgG3 (8C) antibody titers. Antibody levels were analyzed using one-way ANOVA (*p<0.05, ***p<0.001, ****p<0.0001) and represented as mean±SD; n=10 mice/group.

FIGS. 9A and B are schematics of plasmids used to create the recombinant virus expressing Dengue virus NS1 (DENV2 NS1) protein. (9A) The pBKK745 plasmid contains the necessary attP site from the Bxb1 mycobacteriophage (in green). Additionally, to ensure bacterial replication and selection, the plasmid also contains an origin of replication (oriE) and the Kanamycin resistance gene (KanR) and promoter (Kan Promoter). The plasmid also contains a cos site, so it is compatible with cosmid cloning and lambda packaging. There is also a gene expression cassette, consisting of a multiple cloning site (MCS.v4) preceded by a CMV promoter and enhancer and followed by a simian virus 40 polyadenylation signal (SV40 polyA). (9B) The DENV2 NS1 gene was amplified using PCR to have AscI and SbfI sticky ends. The pBKK745 plasmid was digested with AscI and SbfI and ligated to the NS1 gene fragment to generate pBKK840. pBKK840 contains a human platelet-derived growth factor receptor transmembrane (PDGFR™, SEQ ID NO:34) domain and a murine IgK secretion sequence (SEQ ID NO:35) to enhance expression and ensure that NS1 would be expressed on the cell surface. Additionally, the NS1 gene had His, hemagglutinin, and c-myc tags.

FIGS. 10A-D show that Δg(GJDI)-2::DENV NS1 is immunogenic. Mice were intramuscularly vaccinated with 5×10⁶ PFU Δg(GJDI)-2::DENV NS1, 5×10⁶ PFU Δg(GJDI)-2::attB, or VD60 lysate and boosted three weeks later. The mice were then bled three weeks later, and the serum was used for ELISA analysis to determine anti-NS1 reactivity. As a positive control, 1 μg/well of 7E11 anti-NS1 antibody was used. The positive control and immunized sera at 10⁻² dilution were analyzed for total IgG (10A), IgG1 (10B), IgG2b (10C), and IgG2c (10D). Antibody levels were analyzed using one-way ANOVA (**p<0.01, ***p<0.001, ****p<0.0001) and represented as mean±SD; n=5 mice/group.

### DETAILED DESCRIPTION

Disclosed herein is a herpes simplex virus vaccine, a viral vector and a gene expression platform based on a herpes simplex virus type, which incorporates an efficient mycobacteriophage Bxb1 serine integrase-mediated recombination system for easy and fast cloning of specific transgenes into the viral vector genome.

In the present disclosure, the HSV can be herpes simplex virus-1 or herpes simplex virus-2.

A “heterologous nucleic acid” as used herein, refers to a nucleic acid sequence or polynucleotide, and in particular a DNA sequence, that originates from a source foreign to the particular host genome, or, if from the same source, is modified from its original form. The heterologous nucleic acid is constructed to comprise one or more functional units not found together in nature and is designed to transfer a nucleic acid (or nucleic acids) to a host genome. Examples include circular, double-stranded, extrachromosomal DNA molecules (plasmid, shuttle plasmid), cosmids (plasmids containing cos sequences from lambda phage), viral genomes comprising heterologous (non-native) nucleic acid sequences, and the like.

The term “gene” refers to a nucleotide sequence associated with a biological function. Thus, a gene includes a coding sequence and/or the regulatory sequence required for its expression. A gene can also include non-coding DNA segments such as regulatory elements that, for example, form recognition sequences for other proteins. A gene can be obtained from a variety of sources, including cloning from a source of interest or synthesizing from known or predicted sequence information, and may include sequences designed to have desired parameters.

A “transgene” or “heterologous gene”, with regard to its presence in a host genome of a herpes simplex virus or vector backbone, refers to a nucleic acid that is not naturally present in the genome or vector backbone, respectively, or, if from the same source, is modified from its original form. The heterologous nucleic acid can be a DNA sequence including a heterologous gene. A heterologous gene is expressed to yield a heterologous polypeptide. The term “stably integrated” refers to a heterologous nucleic acid that is incorporated into a host genome, replicates as the host genome replicates, and is transferred to progeny. In the present disclosure, the host genome is an HSV (e.g., HSV-2) genome, and the heterologous nucleic acid is integrated into the HSV genome and passed to progeny virus and/or virions. A “heterologous protein” is a protein encoded by a heterologous gene.

The terms “polypeptide,” “peptide” and “protein” are used herein interchangeably to refer to a molecule formed from the linking, in a defined order, of at least two amino acids. The link between one amino acid residue and the next is an amide bond and is sometimes referred to as a peptide bond.

As used herein an “antigen” or “antigenic polypeptide” or “antigenic protein” refers to a polypeptide capable of inducing an immune response, e.g., a humoral and/or cellular mediated response, to the target of interest in a subject. A heterologous gene encoding a polypeptide can thus encode an antigen to induce an immune response to the target of interest e.g., virus, bacterium, parasite, cancer.

A “polynucleotide” or “nucleic acid” or “nucleotide sequence” refers to a polymeric form of nucleotides.

As used herein, “expression cassette” is a recombinant nucleic acid molecule comprising at least one nucleotide sequence of interest operably linked with at least a control sequence (e.g., a promoter). The nucleotide sequence of interest can be, for example but not limited thereto, a heterologous gene encoding a heterologous antigen, a gene encoding a selectable marker (reporter gene), or a combination thereof.

A nucleotide sequence is “operably linked” when placed into a functional relationship with another nucleotide sequence. For example, a nucleotide sequence for a promoter is operably linked to a coding sequence if it stimulates the transcription of the sequence. In general, nucleotide sequences that are operably linked are contiguous, and in the case of a signal sequence both contiguous and in reading phase. However, enhancers, for example, need not be contiguous with the coding sequences whose transcription they control. Linking is accomplished by ligation at convenient restriction sites or at adapters or linkers inserted in lieu thereof.

The term “promoter” refers to a nucleotide sequence which is sufficient to direct transcription of a particular gene. The promoter includes the core promoter, which is the minimal portion of the promoter required to properly initiate transcription and can also include regulatory elements such as transcription factor binding sites. The regulatory elements may promote transcription or inhibit transcription. Regulatory elements in the promoter can be binding sites for transcriptional activators or transcriptional repressors. A promoter can be constitutive or inducible.

“Recombination sites” or “recombination sequences” are specific polynucleotide sequences that are recognized by the recombinase/integrase enzymes described herein. Typically, two different sites are involved (termed “complementary sites”), one present in the target nucleic acid and another on the nucleic acid that is to be integrated at the target recombination site. The terms “attB” and “attP” refer to attachment (or recombination) sites of the Bxb1 integration system and are originally from a bacterial target (e.g., Mycobacterium smegmatis) and a phage donor (Bxb1 mycobacteriophage), respectively. The attB site is also referred to herein as the Bxb1 attB site. The recombination sites can include left and right arms separated by a core or spacer region. Upon recombination between the attB and attP sites, and concomitant integration of a nucleic acid at the target, the recombination sites that flank the integrated DNA are referred to as “attL” and “attR.”

“Codon optimization” is defined as modifying a nucleic acid sequence for enhanced expression in the cells of interest, e.g. human, by replacing at least one, more than one, or a significant number, of codons of the native sequence with codons that are more frequently or most frequently used in the genes of the target species. Various species exhibit particular bias for certain codons of a particular amino acid. In one aspect, the present disclosure relates to codon optimized inserts, nucleic acids or vectors, or host cells comprising such.

The terms “genetically engineered” or “genetically modified” can be used interchangeably and refer to the deliberate modification of the characteristics of an organism (e.g., a virus) by manipulating its genetic material. The genetic engineering or modification can include, for example, the addition, deletion, or rearrangement of one or more polynucleotide sequences.

The term “recombinant” indicates that the genetic material of the organism (e.g., virus) has been genetically engineered.

A “mycobacteriophage” is a phage capable of infecting one or more Mycobacterium strains.

As used herein the complete deletion of a gene from the genome of a virus refers to the removal of at least 98%, at least 99%, or 100% of the nucleic acid sequence encoding the gene from the viral genome.

“Complementing cell” or “complementing cells” or “complementing cell line” refers to Vero cells which express one or more HSV-1 or HSV-2 glycoprotein G, J, D, and I, on a lipid bilayer and phenotypically complements HSV strains deleted for one or more of these glycoproteins. A “non-complementing cell” is thus a cell which does not express any of the one or more HSV glycoproteins G, J, D, and I, and does not phenotypically complement HSV deleted for one or more of these proteins. In one aspect, the complementing cell is a recombinantly engineered Vero cell expressing an HSV-1 gD encoding gene that is able to complement a HSV-2 virus with a glycoprotein D deletion, or a deletion of glycoproteins G, J, D, and I, such that the virus can replicate. In one aspect, the cell is a recombinantly engineered cell comprising one or more heterologous nucleic acid encoding one or more HSV-1 glycoproteins G, J, D, or I and devoid of DNA sequences homologous to sequences present in the recombinant HSV-2 having a deletion of HSV-2 glycoproteins G, J, D, or I, permitting replication of the recombinant HSV-2. In yet another aspect, minimally required DNA sequences for complementation include the HSV-1 gD protein coding sequences, minimal promoter sequences, and a polyadenylation signal. In one aspect, the minimal promoter sequence comprises or is the sequence 5′ATCCCCTAAGGGGGAGGGGCCATTTTACGAGGAGGAGGGGTATAACAAAGTCT GTCTTTAAAAAGCAGGGGTTAGGGAGTTGTTCGGTCATAAGCTTCAGCGCGAAC GACCAACTACCCCGATCATCAGTTATCCTTAAGGTCTCTTTTGTGTGGTGCGTTCC GGT 3′, identified in SEQ ID NO: 36, based on the HSV-1 17+ strain, and described in Roger, J. Watson, 1983, Gene 26, 307-312. gD promoter sequences from other strains of HSV-1 can be used. In another aspect, the cell is a Vero cell. In yet another aspect, the engineered complementing cell is VerB::gD1, a Vero cell containing minimally required DNA sequences for gD complementation. In yet another aspect, the engineered complementing cell is VerB::gD1.6C, a Vero cell containing a minimal promoter comprising the sequence 5′ATCCCCTAAGGGGGAGGGGCCATTTTACGAGGAGGAGGGGTATAACAAAGTCT GTCTTTAAAAAGCAGGGGTTAGGGAGTTGTTCGGTCATAAGCTTCAGCGCGAAC GACCAACTACCCCGATCATCAGTTATCCTTAAGGTCTCTTTTGTGTGGTGCGTTCC GGT 3′, identified in SEQ ID NO:36, and minimally required DNA sequences for gD complementation under control of a minimal promoter with a 5′ 3-nucleotide modification having the sequence 5′CGAATCCCCTAAGGGGGAGGGGCCATTTTACGAGGAGGAGGGGTATAACAAA GTCTGTCTTTAAAAAGCAGGGGTTAGGGAGTTGTTCGGTCATAAGCTTCAGCGCG AACGACCAACTACCCCGATCATCAGTTATCCTTAAGGTCTCTTTTGTGTGGTGCGT TCCGGT 3′, identified in SEQ ID NO:37, based on the HSV-1 17+ strain.

Described herein is an isolated, recombinant herpes simplex virus-2 (HSV-2), Δg(GJDI)-2, having a complete deletion of a region of the genome containing the US4, US5, US6, and US7 genes encoding glycoprotein G, glycoprotein J, glycoprotein D, and glycoprotein I, respectively and shown to be effective as a composition for immunizing, vaccinating, or treating a subject against HSV-1, HSV-2 infection or co-infection.

In an embodiment, an isolated, recombinant herpes simplex virus-2 (HSV-2) having a complete deletion of an HSV-2 glycoprotein G-encoding gene, glycoprotein J-encoding gene, glycoprotein D-encoding gene, and glycoprotein I-encoding gene in the genome for use as a gene expression platform based on a genetically modified herpes simplex virus (HSV) vector. The Δg(GJDI)-2 incorporates an efficient Bxb1 integrase-mediated recombination system for easy and fast cloning of specific transgenes into the genetically modified HSV genome to create an HSV expression vector. The attB sequence of M. smegmatis was inserted into the deleted region of the Δg(GJDI)-2 genome, generating an Δg(GJDI)-2::attB viral vector. The incorporation of the attB sequence in the HSV-2 genome acts as an insertion site for a heterologous nucleic acid containing the specific phage attachment (attP) sequence. This allows the Δg(GJDI)-2::attB to act as a vector backbone for integration of a heterologous gene mediated by the Bxb1 integrase. The HSV vector can be used as a vaccine vector for generating an immune response to an antigen in a subject, or it can be used as a gene expression platform for medical or scientific applications.

The Bxb1 integration system is comprised of: (1) a 38-base pair attB nucleotide sequence from Mycobacterium smegmatis (GGCTTGTCGACGACGGCGGTCTCCGTCGTCAGGATCAT; SEQ ID NO: 1), (2) a 48-base pair attP nucleotide sequence from mycobacteriophage Bxb1 (GGTTTGTCTGGTCAACCACCGCGGTCTCAGTGGTGTACGGTACAAACC; SEQ ID NO: 2), and (3) mycobacteriophage Bxb1 serine integrase. The Bxb1 integration system has been demonstrated to be a highly efficient recombination system that only requires the Bxb1 serine integrase enzyme to initiate and complete recombination between its target attB and attP sites. (Xu, et al., BMC Biotechnol, 2013, vol. 13, p. 87).

The serine integrase, synthesized by the Bxb1 mycobacteriophage, mediates recombination between the phage (attP) and bacterial (attB) attachment sites, which are non-identical. Each of the attB and attP target sites contain an integration core flanked by inverted repeats. The recombination facilitated by Bxb1 serine integrase results in genetic modification at the attB target site. Specifically, recombination between attB and attP results in the formation of hybrid sites attL (left) and attR (right) that cannot be recombined by Bxb1 integrase without additional components. To mediate excision of a previously integrated nucleic acid sequence, another phage-encoded protein called the recombination directionality factor (RDF) is needed in addition to the integrase. However, in the presence of the serine integrase alone without the RDF, the integration reaction is unidirectional and does not require host cofactors.

The attB and/or attP site can be modified, for example by mutation of their respective core sequences, to increase recombination efficiency and/or increase binding affinity of the Bxb1 serine integrase. In an aspect, the attB site has a nucleic acid sequence which is 95%, or 98%, or 99%, or 100% homologous to SEQ ID NO:1. In an aspect, the attP site has a nucleic acid sequence which is 95%, or 98%, or 99%, or 100% homologous to SEQ ID NO:2.

As a recombination partner, a heterologous nucleic acid containing the attP sequence is used to integrate a transgene expression cassette (an expression cassette including a gene encoding a heterologous antigen or other protein) into the genome of the Δg(GJDI)-2::attB. The heterologous nucleic acid can be, for example, a shuttle plasmid. To facilitate site-specific integration of the heterologous nucleic acid harboring the transgene expression cassette into the genomic DNA of Δg(GJDI)-2::attB, an in vitro reaction is carried out by combining the heterologous nucleic acid with the genomic DNA of the Δg(GJDI)-2::attB in the presence of the Bxb1 integrase. Recombination between attB and attP results in the formation of hybrid sites attL and attR. Thus, following integration of the plasmid at the attB site in the genome of the Δg(GJDI)-2::attB, the HSV-2 genome comprises an attL sequence at one end (e.g., the 5′ end or the 3′ end) of the integrated expression cassette and an attR sequence at the other end (e.g., the 3′ end or the 5′ end) of the integrated expression cassette. It has been discovered that the Bxb1 integrase-mediated recombination system disclosed herein efficiently and stably inserts up to 6 kilobases (kB) of heterologous nucleic acid sequences into the Δg(GJDI)-2 viral genome, and thus allows a high-throughput cloning of vaccine antigens for the rapid development of antigen testing and vaccine development. Additionally, this high-throughput cloning system can be used to express heterologous proteins for the application of heterologous gene expression in in vivo and in vitro systems. Importantly, Bxb1 integrase-mediated recombination is a powerful system for rapid, site-specific, high-throughput cloning of transgenes into the HSV viral genome.

The present disclosure provides a genetically modified herpes simplex virus (HSV) comprising a genome comprising a complete deletion of glycoprotein D-encoding gene and a recombination sequence inserted in a region of the genome from which the glycoprotein D-encoding gene has been deleted. The recombination sequence comprises an attB sequence from Mycobacterium smegmatis.

In an aspect, the HSV genome comprises a complete deletion of a region comprising the glycoprotein D-encoding gene. In an aspect, the genome comprises a complete deletion of a region comprising the glycoprotein G-encoding gene, the glycoprotein J-encoding gene, the glycoprotein D-encoding gene, and the glycoprotein I-encoding gene.

The HSV genome thus comprises a complete deletion of the glycoprotein G-encoding gene, the glycoprotein J-encoding gene, and the glycoprotein I-encoding gene in addition to the deletion of the glycoprotein D-encoding gene.

In the present disclosure, the HSV can be herpes simplex virus-1 (HSV-1) or herpes simplex virus-2 (HSV-2).

In an aspect, the present disclosure provides a genetically modified HSV-1 or HSV-2 comprising a complete deletion of the glycoprotein G-encoding gene, the glycoprotein J-encoding gene, the glycoprotein D-encoding gene, and the glycoprotein I-encoding gene in the HSV-1 or HSV-2 genome; and a recombination sequence inserted in a region of the HSV-1 or HSV-2 genome from which the glycoprotein G-encoding gene, the glycoprotein J-encoding gene, the glycoprotein D-encoding gene, and the glycoprotein I-encoding gene have been deleted, wherein the recombination sequence comprises an attB sequence from Mycobacterium smegmatis.

The genetically modified HSV is generated by deleting each of the following genes from the genome of HSV through homologous recombination: glycoprotein G-encoding gene (US4 gene), glycoprotein J-encoding gene (US5 gene), glycoprotein D-encoding gene (US6), and glycoprotein I-encoding gene (US7 gene). These four genes are collectively referred to as “glycoprotein G, J, D, and I-encoding genes”. The glycoprotein G, J, D, and I-encoding genes are adjacent to one another in the HSV genome and can be deleted from the HSV genome sequentially or simultaneously. In an aspect, the glycoprotein G, J, D, and I-encoding genes were deleted from the HSV genome simultaneously.

In an aspect, the HSV glycoprotein G-encoding gene is an equivalent of the HSV-1 or HSV-2 US4 gene, the glycoprotein J-encoding gene is an equivalent of the HSV-1 or HSV-2 US5 gene, the glycoprotein D gene is an equivalent of the HSV-1 or HSV-2 US6 gene, and/or the glycoprotein I gene is an equivalent of the HSV-1 or HSV-2 US7 gene. Such equivalents are easily identifiable by those of skill in the art using readily available sequencing and alignment tools.

In an aspect, the glycoprotein G-encoding gene is HSV-2 US4 gene, the glycoprotein J-encoding gene is HSV-2 US5 gene, the glycoprotein D-encoding gene is HSV-2 US6 gene, and the glycoprotein I-encoding gene is HSV-2 US7 gene. In an aspect, the glycoprotein G-encoding gene is HSV-1 US4 gene, the glycoprotein J-encoding gene is HSV-1 US5 gene, the glycoprotein D-encoding gene is HSV-1 US6 gene, and the glycoprotein I-encoding gene is HSV-1 US7 gene.

In an aspect, the genetically modified HSV is HSV-2. The HSV-2 glycoprotein G (gG) comprises the amino acid sequence set forth in SEQ ID NO: 3.

(SEQ ID NO: 3)
MHAIAPRLLLLFVLSGLPGTRGGSGVPGPINPPNNDVVFPGGSPVAQYCYAYPRLDD
PGPLGSADAGRQDLPRRVVRHEPLGRSFLTGGLVLLAPPVRGFGAPNATYAARVTY
YRLTRACRQPILLRQYGGCRGGEPPSPKTCGSYTYTYQGGGPPTRYALVNASLLVPI
WDRAAETFEYQIELGGELHVGLLWVEVGGEGPGLTAPPQAARAEGGPCVPPVPAGR
PWRSVPPVWYSAPNPGFRGLRFRERCLPPQTPAAPSDLPRVAFAPQSLLVGITGRTFIR
MARPTEDVGVLPPHWAPGALDDGPYAPFPPRPRFRRALRTDPEGVDPDVRAPRTGR
RLMALTENASSDSPTSAPEKTPLPVSATAMAPSVDPSAEPTAPATTTPPDEMATQAAT
VAVTPEETAVASPPATASVESSPLPAAAATPGAGHTNTSSASAAKTPPTTPAPTTPPPT
STHATPRPTTPGPQTTPPGPATPGPVGASAAPTADSPLTASPPATAPGPSAANVSVAA
TTATPGTRGTARTPPTDPKTHPHGPADAPPGSPAPPPPEHRGGPEEFEGAGDGEPPED
DDSATGLAFRTPNPNKPPPARPGPIRPTLPPGILGPLAPNTPRPPAQAPAKDMPSGPTP
QHIPLFWFLTASPALDILFIISTTIHTAAFVCLVALAAQLWRGRAGRRRYAHPSVRYV
CLPPERD

The HSV-2 glycoprotein J (gJ) comprises the amino acid sequence set forth in SEQ ID NO:4.

(SEQ ID NO: 4)
MDRYAVRTWGIVGILGCAAVGAAPTGPASDTTNATARLPTHPPLIRSGG
FAVPLIVGGLCLMILGMACLLEVLRRLGRELARCCPHAGQFAP

The HSV-2 glycoprotein D (gD) comprises the amino acid sequence set forth in SEQ ID NO:5.

(SEQ ID NO: 5)
MGRLTSGVGTAALLVVAVGLRVVCAKYALADPSLKMADPNRFRGKNLPVLDQLTD
PPGVKRVYHIQPSLEDPFQPPSIPITVYYAVLERACRSVLLHAPSEAPQIVRGASDEAR
KHTYNLTIAWYRMGDNCAIPITVMEYTECPYNKSLGVCPIRTQPRWSYYDSFSAVSE
DNLGFLMHAPAFETAGTYLRLVKINDWTEITQFILEHRARASCKYALPLRIPPAACLT
SKAYQQGVTVDSIGMLPRFIPENQRTVALYSLKIAGWHGPKPPYTSTLLPPELSDTTN
ATQPELVPEDPEDSALLEDPAGTVSSQIPPNWHIPSIQDVAPHHAPAAPSNPGLIIGAL
AGSTLAVLVIGGIAFWVRRRAQMAPKRLRLPHIRDDDAPPSHQPLFY

The HSV-2 glycoprotein I (gI) comprises the amino acid sequence set for in SEQ ID NO: 6.

(SEQ ID NO: 6)
MPGRSLQGLAILGLWVCATGLVVRGPTVSLVSDSLVDAGAVGPQGFVEEDLRVFGE
LHFVGAQVPHTNYYDGIIELFHYPLGNHCPRVVHVVTLTACPRRPAVAFTLCRSTHH
AHSPAYPTLELGLARQPLLRVRTATRDYAGLYVLRVWVGSATNASRFVLGVALSAN
GTFVYNGSDYGSCDPAQLPFSAPRLGPSSVYTPGASRPTPPRTTTPPSSPRDPTPAPGD
TGTPAPASGESAPPNSTRSASESRHRLTVAQVIQIAIPASIIAFVFLGSCICFIHRCORRY
RRPRGQIYNPGGVSCAVNEAAMARLGAELRSHPNTPPKPRRRSSSSTTMPSLTSIAEE
SEPGPVVLLSVSPRPRSGPTAPQEV

The above amino acid sequences for the glycoproteins G, J, D, and I (SEQ ID NOs: 3-6, respectively) were translated from the short gun sequencing (Miseq, Illumina, USA) of HSV-2 strain G. These sequences are published in Chang et al., 2022, Viruses 14, 536.

In a method of making the genetically modified HSV-2, the genome of unmodified HSV-2 (strain G) is partially digested into large fragments with BamHI and ligated into the cosmid pYUB328 having arms made with BclII and NheI. The ligated fragments were generated via lambda packaging and a cosmid (pYUB2156) containing a 40-kB fragment around the US6 gene (gD-encoding gene) was selected. The selected cosmid contains the HSV-2 US3, US4, US5, US6, US7, and US8 genes, at minimum. Following cosmid amplification, the US4, US5, US6, and US7 genes are deleted by restriction digest to generate a new cosmid (pBRL951) including the non-deleted (wild type) portion of the original 40-kB fragment. Linearized pBRL951 is co-transfected with DNA from a ΔgD-2 strain including a gene encoding a fluorescent protein (e.g., a red fluorescent protein [RFP] or green fluorescent protein [GFP]) under the control of a promoter, into complementing cells expressing HSV-1 gG, gD, gJ, and gI on the lipid bilayer of the cell membrane. Within the complementing cells, HSV-2 virus/virions is produced which express HSV-1 gG, gD, gJ, and gI on the viral envelope.

In an aspect, the ΔgD-2 including a gene encoding the fluorescent protein is the strain ΔgD-2::RFP. The ΔgD-2::RFP is a genetically modified HSV-2 having a partial deletion of glycoprotein D encoding gene (US6) such that only glycoprotein D coding sequence was deleted. The deleted portion of the glycoprotein D gene in the ΔgD-2::RFP is replaced with a gene encoding a red fluorescent protein (RFP) under control of a elongation factor-α (EF1a) promoter with a Woodchuck Hepatitis Virus Posttranscriptional Regulatory Element (WPRE) and polyA tail. Within the complementing cells, homologous recombination occurs between the US3 and US8 gene regions in the linearized plasmid pBRL951 and the ΔgD-2::RFP genome. Successful recombination leads to the loss of the RFP gene and generation of Δg(GJDI)-2. Δg(GJDI)-2 virus or virions are isolated by selecting cell culture plaques which are negative for fluorescent protein expression and then plaque purified over 3 generations. The genetically modified HSV-2 having a deletion of the glycoprotein G, J, D, and I-encoding genes is referred to herein as Δg(GJDI)-2.

In the methods disclosed herein, the complementing cells (also referred to as a complementing cell line) are VD60 cells which express HSV-1 gG, gJ, gD, and gI on a lipid bilayer and phenotypically complement for the deleted HSV-2 glycoproteins G, J, D, I. (See, e.g., Ligas et al., J Virol. 1988 May; 62(5):1486-94, hereby incorporated by reference) Δg(GJDI)-2 virus produced by co-transfection of the linearized plasmid pBRL951 and DNA from ΔgD-2::RFP in the complementing cells, is phenotypically complemented with the HSV-1 gD, gG, gJ and gI proteins produced by the complementing cell. The phenotypically complemented Δg(GJDI)-2 express HSV-1 gG, gD, gJ, and gI on the viral envelope. The Δg(GJDI)-2 is a single cycle virus in non-complementing cells.

Δg(GJDI)-2 harboring the attB site is constructed by inserting the attB sequence from M. smegmatis into the region of the Δg(GJDI)-2 genome from which the glycoprotein G, J, D, and I-encoding genes have been deleted. In a method disclosed herein, the attB sequence of the Bxb1 mycobacteriophage is amplified by PCR and cloned into the pBRL951 plasmid between the flanking US3 and US8 gene sequences to produce the plasmid pBRL962. The linearized pBRL962 is co-transfected into complementing (VD60) cells with ΔgD-2 viral DNA including a gene encoding a fluorescent protein (e.g., ΔgD-2::RFP), and VD60 cell culture plaques negative for RFP expression are selected and plaque purified over 3 generations. The attB sequence is inserted in the HSV-2 genome between a sequence encoding HSV-2 US3 gene and a sequence encoding HSV-2 US8 gene. The resulting genetically modified HSV-2 Δg(GJDI)-2 including the attB sequence is designated as Δg(GJDI)-2::attB or Δg(GJDI)-2 attB. In non-complementing cell lines, the Δg(GJDI)-2::attB is a single cycle virus.

In an embodiment, the genetically modified HSV-2 Δg(GJDI)-2 or Δg(GJDI)-2::attB virus or virion as described herein is provided in a vaccine composition. In an embodiment, the vaccine composition comprises an immunological adjuvant.

Also provided is a pharmaceutical composition comprising the virus or virion as described herein, and a pharmaceutically acceptable carrier.

Disclosed herein also are methods of eliciting an immune response to HSV-1 or HSV-2 in a subject. In an aspect, the method comprises administering the HSV-2 virus or virion disclosed herein in an amount effective to elicit the immune response to HSV-1 or HSV-2 or a co-infection of HSV-1 and HSV-2 in the subject. The vaccine elicits an immune response comprising neutralizing antibodies and/or nonneutralizing antibodies able to activate effector immune functions in immune cells, such as antibody-dependent cellular cytotoxicity (ADCC). Without being bound to a theory, nonneutralizing antibodies stimulate effector cell mechanisms, including antibody-mediated phagocytosis and antibody-dependent cellular cytotoxicity (ADCC), both of which require activation of the Fcγ receptors (FcγRs). ADCC is initiated by immune cells, including killer cells and macrophages, when an Fc receptor is engaged by the Fc region of an antibody. This interaction activates a downstream signaling cascade that results in a cytotoxic response against the infected target cell. Among FcγRs, FcγRIV seems to have a central role in mediating ADCC. Specific isotypes of IgG antibodies are associated with binding and modulation of FcγR and subsequent ADCC activation, including the IgG1 and IgG3 subtypes in humans, as well as IgG2a and IgG2c subtypes in mice. ADCC-mediated antibodies have been demonstrated to protect against virus challenge by passive transfer of immune sera from vaccinated to nonvaccinated subjects.

Also provided is a method of treating an HSV-1 infection, or HSV-1 and HSV-2 co-infection, in a subject, or treating a disease caused by an HSV-2 infection or HSV-1 and HSV-2 co-infection in a subject comprising administering to the subject an amount of (i) a Δg(GJDI)-2 or Δg(GJDI)-2::attB virus as described herein; (ii) a Δg(GJDI)-2 or Δg(GJDI)-2::attB virion as described herein, (iii) a Δg(GJDI)-2 or Δg(GJDI)-2::attB vaccine as described herein; (iv) a Δg(GJDI)-2 or Δg(GJDI)-2::attB composition as described herein; or (v) a Δg(GJDI)-2 or Δg(GJDI)-2::attB pharmaceutical composition as described herein, in an amount effective to treat an HSV-2 infection or treat a disease caused by an HSV-2 infection in a subject or an amount effective to treat an HSV-1 and HSV-2 co-infection or treat a disease caused by an HSV-1 and HSV-2 co-infection in a subject.

Also provided is a method of vaccinating a subject for an HSV-1 infection, or HSV-1 and HSV-2 co-infection, comprising administering to the subject an amount of (i) a Δg(GJDI)-2 or Δg(GJDI)-2::attB virus as described herein; (ii) a Δg(GJDI)-2 or Δg(GJDI)-2::attB virion as described herein, (iii) a Δg(GJDI)-2 or Δg(GJDI)-2::attB vaccine as described herein; (iv) a Δg(GJDI)-2 or Δg(GJDI)-2::attB composition as described herein; or (v) a Δg(GJDI)-2 or Δg(GJDI)-2::attB pharmaceutical composition as described herein, in an amount effective to vaccinate a subject for an HSV-1 infection, or HSV-1 and HSV-2 co-infection.

Also provided is a method of immunizing a subject against an HSV-1 infection, or HSV-1 and HSV-2 co-infection, comprising administering to the subject an amount of (i) a Δg(GJDI)-2 or Δg(GJDI)-2::attB virus as described herein; (ii) a Δg(GJDI)-2 or Δg(GJDI)-2::attB virion as described herein, (iii) a Δg(GJDI)-2 or Δg(GJDI)-2::attB vaccine as described herein; (iv) a Δg(GJDI)-2 or Δg(GJDI)-2::attB composition as described herein; or (v) a Δg(GJDI)-2 or Δg(GJDI)-2::attB pharmaceutical composition as described herein, in an amount effective to immunize a subject against an HSV-1 infection, or HSV-1 and HSV-2 co-infection.

In an embodiment of the methods herein for immunizing, vaccinating or eliciting an immune response, passive transfer of the virion or virus or the antibodies or immune factors induced thereby may be effected from one subject to another. The relevant product may be treated after obtention from one subject before administration to a second subject. In a preferred embodiment of the inventions described herein, the subject is a mammalian subject. In an embodiment, the mammalian subject is a human subject.

HSV-2 and HSV-1 diseases are known in the art, and are also described herein. Both treatment and prevention of HSV-2 and HSV-1 diseases are each separately encompassed. Also treatment or prevention of a HSV-2 and HSV-1 co-infection are covered. Prevention is understood to mean amelioration of the extent of development of the relevant disease or infection in a subject treated with the virus, virion, vaccine or compositions described herein, as compared to an untreated subject.

In an embodiment, the genetically modified HSV including the attB recombination sequence (e.g., Δg(GJDI)-2::attB) provides a vector backbone into which a heterologous nucleic acid can be inserted or integrated. A “vector backbone” refers to a nucleic acid molecule capable of transporting one or more other nucleic acids to which it has been linked. The vector backbone comprises one or more nucleic acid sequences that are naturally present in the HSV genome sequence, but the complete sequence is not identical to the naturally occurring HSV genome sequence. In an aspect, the vector backbone is made by complete deletion of at least the glycoprotein D encoding gene from the HSV genome and the subsequent insertion of the attB site in the region from which it was deleted. In an aspect, the vector backbone is made by complete deletion of the glycoprotein G, J, D, I-encoding genes from the genome of HSV and the subsequent insertion of the attB site in the region of the genome from which they were deleted. Accordingly, an HSV vector backbone includes those sequences both directly derived from the naturally occurring HSV strain and those which are subsequently derived. In an aspect, the HSV is HSV-1 or HSV-2. In an aspect, the HSV is HSV-1. In an aspect, the HSV is HSV-2.

The present disclosure also provides a HSV gene expression vector or HSV vaccine vector.

In an aspect, a HSV gene expression vector comprises an HSV genome comprising: a complete deletion of glycoprotein G-encoding gene, glycoprotein D-encoding gene, glycoprotein J-encoding gene, and glycoprotein I-encoding gene; a heterologous nucleic acid comprising an expression cassette and inserted in a region of the HSV genome from which the glycoprotein G-encoding gene, glycoprotein D-encoding gene, glycoprotein J-encoding gene, and glycoprotein I-encoding gene have been deleted; an attL sequence; and an attR sequence; wherein the attL sequence is adjacent to a first end of the expression cassette and the attR sequence is adjacent to a second end of the expression cassette, and wherein the expression cassette comprises in operable communication a promoter and at least one gene encoding a heterologous protein.

The HSV gene expression vector can be a HSV vaccine vector. In an aspect, an HSV vaccine vector comprises an HSV genome comprising: a complete deletion of glycoprotein G-encoding gene, glycoprotein D-encoding gene, glycoprotein J-encoding gene, and glycoprotein I-encoding gene; a heterologous nucleic acid comprising an expression cassette and inserted in a region of the HSV genome from which the glycoprotein G-encoding gene, glycoprotein D-encoding gene, glycoprotein J-encoding gene, and glycoprotein I-encoding gene have been deleted; an attL sequence; and an attR sequence; wherein the attL sequence is adjacent to a first end of the expression cassette and the attR sequence is adjacent to a second end of the expression cassette, and wherein the expression cassette comprises in operable communication a promoter and at least one gene encoding a heterologous antigen.

In an aspect, the HSV gene expression vector is an HSV-2 gene expression vector or an HSV-1 gene expression vector. In an aspect, the HSV vaccine vector is an HSV-2 vaccine vector or an HSV-1 vaccine vector.

Also disclosed is a method of producing an HSV gene expression vector comprising a gene encoding a heterologous protein, e.g. antigen. The method disclosed herein comprises: providing a genetically modified HSV-2 comprising a genome comprising a complete deletion of glycoprotein G-encoding gene, glycoprotein J-encoding gene, glycoprotein D-encoding gene, and glycoprotein I-encoding gene, and a first recombination sequence inserted in a region of the genome from which the glycoprotein G-encoding gene, glycoprotein J-encoding gene, glycoprotein D-encoding gene, and glycoprotein I-encoding gene have been deleted; providing a heterologous nucleic acid comprising a second recombination sequence and an expression cassette comprising the gene encoding the heterologous protein; contacting the HSV genome and the heterologous nucleic acid in the presence of a bacteriophage Bxb1 serine integrase under conditions in which sequence-specific recombination occurs between the first recombination sequence and the second recombination sequence to provide a recombined HSV genome; and transfecting a complementing cell with the recombined HSV genome to obtain the HSV gene expression vector, wherein the first recombination sequence comprises an attB sequence from Mycobacterium smegmatis and the second recombination sequence comprises an attP sequence from bacteriophage Bxb1.

The HSV gene expression vector and the method of producing the HSV gene expression vector are discussed in further detail below.

The HSV gene expression vector is constructed from the above-described genetically modified HSV having an HSV genome including a complete deletion of the glycoprotein G-encoding gene, glycoprotein D-encoding gene, glycoprotein J-encoding gene, and glycoprotein I-encoding gene, and a recombination sequence inserted in the region of the HSV genome from which the genes have been deleted. As a recombination partner, a heterologous nucleic acid (e.g., shuttle plasmid) containing the attP site upstream or downstream of a transgene expression cassette is used. The heterologous nucleic acid is inserted in the HSV vector backbone within the region from which the genes have been deleted. The heterologous nucleic acid is inserted by site-specific integration into the genome at the attB site. Site specific integration occurs by contacting the heterologous nucleic acid including the attP site with the HSV genomic DNA including the attB site in the presence of the Bxb1 serine integrase. Recombination between attB and attP results in the formation of hybrid sites attL and attR in the recombined HSV genome. The genome of the resulting HSV vaccine vector thus comprises an attL sequence at one end of the integrated expression cassette and an attR sequence at the other end of the integrated expression cassette. In an aspect, the attL is at a 5′ end and the attR is at a 3′ end of the integrated expression cassette. In another aspect, the attL is at a 3′ end and the attR is at a 5′ end of the integrated expression cassette.

In an aspect, the HSV gene expression vector is a vaccine vector and the heterologous protein is an antigen. In an aspect the HSV gene expression vector is an HSV-1 or an HSV-2 gene expression vector. In an aspect, one or more heterologous antigen is expressed by the vaccine vector.

In an aspect the HSV gene expression vector is an HSV-2 vaccine vector. The HSV-2 gene expression vector is constructed from the Δg(GJDI)-2::attB. A heterologous nucleic acid (e.g., shuttle plasmid) containing the attP site upstream of a transgene expression cassette is used as the recombination partner. The insertion site of the heterologous nucleic acid in the Δg(GJDI)-2::attB vector backbone is within the region from which the glycoprotein G,J,D,I-encoding genes have been deleted. The heterologous nucleic acid is inserted by site-specific integration in the genome of Δg(GJDI)-2::attB at the attB site. Site specific integration occurs by contacting the heterologous nucleic acid including the attP site with the genomic DNA of the Δg(GJDI)-2::attB in the presence of the Bxb1 serine integrase. Following integration of the plasmid at the attB site in the genome of the Δg(GJDI)-2::attB, the HSV-2 genome comprises an attL sequence at one end of the integrated expression cassette and an attR sequence at the other end of the integrated expression cassette. In an aspect, the attL is at a 5′ end and the attR is at a 3′ end of the integrated expression cassette. In another aspect, the attL is at a 3′ end and the attR is at a 5′ end of the integrated expression cassette.

In an aspect, the attL site or sequence has a nucleic acid sequence of GGC TTGTCGACGACGGCGGTCTCAGTGGTGTACGGTACAAACC (SEQ ID NO: 7). In an aspect, the attR site or sequence has a nucleic acid sequence of GGTTTGTCTGGTCAACCACCGCGGTCTCCGTCGTCAGGATCAT (SEQ ID NO: 8).

Following site-specific integration of the heterologous nucleic acid in the HSV vector backbone (e.g., Δg(GJDI)-2::attB vector backbone), the recombined HSV genome is transfected into the VD60 complementing cell line to obtain the HSV gene expression vector. Since the HSV gene expression vector is cultured in the complementing VD60 cells expressing HSV-1 glycoproteins G, J, D, and I, the HSV-2 gene expression vector is phenotypically complemented with the HSV-1 glycoproteins G, J, D, and I on a lipid bilayer of the virus, i.e. the viral envelope. The HSV gene expression vector is thus a single cycle virus in a non-complementing cell lines.

The HSV vaccine vector disclosed herein comprises a heterologous nucleic acid inserted in the HSV genome. In an aspect, the heterologous nucleic acid is constructed from a plasmid including the attP sequence inserted upstream of a promoter, a multiple cloning site (MCS), and a polyadenylation signal sequence; with or without the inclusion of a WPRE upstream of the polyadenylation signal sequence. The gene encoding the heterologous protein is amplified by PCR or isolated with restriction digest and inserted into the MCS of the constructed plasmid to be in operable communication with the promoter. In an aspect, the heterologous nucleic acid inserted in the HSV genome comprises an expression cassette, which is comprised of at least one gene encoding a heterologous protein in operable communication with a promoter and a polyadenylation signal sequence.

Other useful elements can also be included in the heterologous nucleic acid. For example, the heterologous nucleic acid further comprises a gene encoding a selectable marker, an antibiotic resistance gene, a bacterial origin of replication, a viral origin of replication, or a combination thereof. The heterologous nucleic acid can also include other sequences such as a targeting or localization sequence, a tag sequence, a self-cleavage peptide (e.g. P2A or T2A), a sequence of a fluorescent protein that is not a component of the expression cassette, or other elements, like a WPRE, that can be used to modulate transgene expression or improve cloning of antigen, like an E. coli cosmid sequence (a cos sequence). Additionally, a transmembrane domain, such as the platelet-derived growth factor receptor (PDGFR) transmembrane domain, the transmembrane domain and cytosolic tail of murine CD80, the glycosylphosphatidylinositol anchor encoded by the C-terminal extension of decay-accelerating factor (DAF), or the transmembrane domain of the H1 subunit of the human asialoglycoprotein receptor (ASGPR), and others know in the art, may be fused to or used in conjunction with a heterologous gene in a gene expression cassette to enable expression of the antigen on the cell membrane surface. In an aspect, the antibiotic resistance gene comprises an ampicillin, a kanamycin, or zeocin resistance gene.

The expression cassette is designed to facilitate the expression of at least one heterologous gene in a host cell when a host cell is infected with the HSV vaccine vector. In an aspect, the host cell is a mammalian cell, and the expression cassette is configured for expression of the encoded genes following infection of the mammalian cell with the HSV-2 vaccine vector. The promoter included in the expression cassette drives the synthesis of a primary transcript. Exemplary promoters include inducible promoters, constitutive promoters, tissue-specific promoters, and synthetic promoters. In an aspect, the promoter comprises a CMV promoter, a tetracycline inducible expression (TRE) promoter, an SV40 promoter, a CAG promoter, a promoter of Elongation Factor 1α gene (P_{EF1α}), or a combination thereof.

Expression of the heterologous gene can be qualitatively and/or quantitatively assessed based on expression of a gene encoding a selectable marker (also referred to herein as “reporter gene” and “reporter protein”). The gene encoding the selectable marker can be inserted upstream and/or downstream of the gene encoding the heterologous polypeptide. In an aspect, the selectable marker gene is inserted downstream of the gene encoding the heterologous polypeptide. In an aspect, the selectable marker is operably linked to the promoter and to the gene encoding the heterologous polypeptide. The selectable marker can induce a visually identifiable characteristic distinguishable from the cell in which it is being expressed and which can be readily measured. Alternatively, the selectable marker can be one which confers a host cell with the ability to grow in the presence of a selective agent that is normally toxic to the host cell, or one which confers a host cell with the ability to grow in the absence of a required nutrient.

Non-limiting examples of genes encoding selectable markers include a gene encoding β-galactosidase (lacZ), a fluorescent protein, a gene encoding a luminescent protein, histidinol dehydrogenase (hisD), a gene encoding antibiotic resistance (e.g., amp^{r}, cam^{r}, tet^{r}, blasticidin^{r}, neo^{r}, hyg^{r}), or a combination thereof. In an aspect, the selectable marker is a reporter protein such as a fluorescent protein, a luminescent protein, or a combination thereof. Non-limiting examples include luciferase, beta-lactamase, alkaline phosphatase, green fluorescent protein (GFP), enhanced green fluorescent protein (EGFP), red fluorescent protein (RFP), monomeric Infrared Fluorescent Protein (mIFP), Long Stokes Shift monomeric Orange (LssmOrange), Tag Red Fluorescent Protein 657 (TagRFP657), monomeric Orange2 (mOrange2), monomeric Apple (mApple), Sapphire, blue fluorescent protein (BFP), monomeric tag blue fluorescent protein (mTagBFP2), tdTomato, monomeric Cherry (mCherry), yellow fluorescent protein (YFP), enhanced yellow fluorescent protein (EYFP), monomeric cerulean3 (mCerulean3), enhanced green fluorescent protein (EGFP), or a combination thereof. Expression of the reporter protein can be detected using any method suitable for detecting and quantifying immunofluorescent cells, for example, methods such as fluorescent microcopy, fluorescence activated cell sorting (FACS), and/or immunofluorescent microscopy. The selectable marker is not limited to those described herein and other suitable selectable markers or selectable marker genes can also be used.

In an aspect, the expression cassette comprises in operable communication the promoter, the at least one gene encoding the heterologous protein, the selectable marker, and a polyadenylation (polyA) signal sequence. The polyA signal sequence promotes polyadenylation and transcription termination and is located downstream of the heterologous gene and the selectable marker gene. Exemplary polyA signal sequences include the SV40 poly(A) signal, the bovine growth hormone polyadenylation signal (bGHpA), human growth hormone polyadenylation signal (hGHpA), and rabbit beta globin polyadenylation signal (rbGlob). However, the polyA signal is not limited thereto.

If desired, the gene encoding the heterologous protein can be codon-optimized for expression.

In an aspect, the expression cassette is designed to facilitate the expression of at least one heterologous gene in a host cell. In an aspect, the expression cassette is designed to facilitate the expression of a plurality of heterologous genes, each encoding a different heterologous protein. In an aspect, the heterologous nucleic acid comprises an expression cassette comprising a plurality of genes, each encoding a different heterologous protein. The plurality of genes can encode heterologous proteins from the same organism or from different organisms. The plurality of heterologous genes are distinct from one another, and each encodes a different heterologous protein. The plurality of heterologous genes can include at least two, at least three, at least four, at least five, at least six, distinct heterologous genes, and is dependent upon the size of the gene. In an aspect, the cloning capacity of the HSV gene expression vector is about 6 kilobases and the number of heterologous genes contained in the expression cassette is 1 to 6. In an aspect, the HSV gene expression vector is Δg(GJDI)-2.

The expression cassette can be designed to facilitate co-expression of multiple separate heterologous proteins from the same mRNA or from different mRNAs. For expression of two or more heterologous proteins from different mRNAs, the expression cassette can include more than one promoter. Each promoter can be operably linked to a single gene encoding a heterologous protein or to multiple genes. To facilitate co-expression of a plurality (e.g., two or more) of separate heterologous proteins from the same mRNA, the expression cassette can include a sequence between individual heterologous genes encoding, for example, a proteolytic cleavage site between individual heterologous genes, an internal ribosome entry site (IRES), or a combination thereof.

Internal ribosome entry sites (IRES) allow translation of the RNAs in a cap-independent manner. The presence of an IRES allows for 2 peptides to be produced from the same mRNA.

A proteolytic cleavage site can include, for example, a 2A peptide, also known as a 2A self-cleaving peptide. The 2A self-cleaving peptides is an 18-22 amino acid long virus-derived polypeptide from the Picornaviridae family, which is used to cleave a longer peptide into shorter peptides in a eukaryotic cell. A GSG residue can be added to the 5′ end of the 2A peptide to improve cleavage efficiency.

| Peptide | Source | Sequence |
|---|---|---|
| T2A | thosea asigna virus 2A | (GSG)EGRGSLLTCGDVEENPGP |
| | | (SEQ ID NO: 13) |
| P2A | porcine teschovirus-1 2A | (GSG)ATNFSLLKQAGDVEENPGP |
| | | (SEQ ID NO: 14) |
| E2A | equine rhinitis A virus | (GSG)QCTNYALLKLAGDVESNPGP |
| | | (SEQ ID NO: 15) |
| F2A | foot-and-mouth disease | (GSG)VKQTLNFDLLKLAGDVESNPGP |
| | virus | (SEQ ID NO: 16) |

2A self-cleaving peptides function by making the ribosome skip the synthesis of a peptide bond at the C-terminus of the 2A peptide, leading to separation between the end of the 2A sequence and the next peptide downstream. It is understood that 2A peptide cleavage occurs between the glycine and proline residues found on the C-terminus of the 2A peptide, and as a result, the upstream peptide will have a few additional residues added to the end, while the downstream peptide will start with the proline.

In an aspect, the expression cassette comprises in operable communication the promoter, a plurality of heterologous genes each encoding a different heterologous antigen, a polyadenylation signal sequence, and a 2A self-cleaving peptide between the plurality of heterologous genes. The expression cassette can also include a selectable marker. In an aspect, the 2A self-cleaving peptide encoded in the expression cassette is a T2A peptide, a P2A peptide, or a combination thereof.

The gene encoding the heterologous protein can be derived from a living organism, comprising for example, a virus, a bacterium, a fungus, a parasite, a plant, a mammalian cell (e.g., a human cell, a non-human animal cell), or a combination thereof. The heterologous protein can be a surface protein or a non-surface protein. In an aspect, the heterologous protein gene comprises a virus gene, a bacterial gene, a fungal gene, a parasite gene, a plant gene, a cancer gene, or a combination thereof. In an aspect, the heterologous protein is a viral antigen, a bacterial antigen, a parasite antigen, a fungal antigen, a plant antigen, a cancer antigen, or a combination thereof.

The heterologous protein can be from a mammalian cell, a non-mammalian cell, or a combination thereof. The heterologous protein can be of clinical or medical benefit or interest, including for example, a protein for the use of cellular modulation or disruption.

The virus can be a pathogenic virus, examples of which include adenovirus, cytomegalovirus (CMV), coxsackie virus, Crimean-Congo hemorrhagic fever virus, chikungunya virus, dengue virus, Dhori virus, Eastern equine encephalitis (EEE) virus, Ebola virus, Epstein Barr virus (EBV), Hanta virus, hepatitis viruses (e.g., hepatitis A, hepatitis B, hepatitis C, hepatitis D, hepatitis E), herpesvirus (e.g., human herpesvirus-6 [HHV-6], HHV-8), human immunodeficiency virus (HIV), human papilloma virus, human SARS corona virus, SARS CoV-2, human T lymphotropic virus (HTLV), influenza virus, Japanese encephalitis virus, Marburg virus, measles virus, mumps virus, poliovirus, Norwalk virus, smallpox, parvovirus, rabies virus, reovirus, rhinovirus, Rift Valley fever virus, rotavirus, rubella virus, severe fever with thrombocytopenia syndrome (SFTS) virus, respiratory syncytial virus (RSV), varicella zoster virus, Western equine encephalitis virus, West Nile virus, yellow fever virus, Zika virus, or a combination thereof.

The bacterium can be a pathogenic bacterium, examples of which include Actinomyces sp, Bacillus sp., Bartonella sp., Bordatella sp., Borellia sp., Brucella sp., Campylobacter sp., Chlamydia sp., Clostridium sp., Corynebacterium sp., Coxiella sp., Enterobacter sp., Enterococcus sp., Escherichia sp., Francisella sp, Gardnerella sp., Haemophilus sp., Helicobacter sp., Klebsiella sp., Legionella sp., Leptospira sp., Listeria sp., Mycobacterium sp., Mycoplasma sp., Neisseria sp., Nocardia sp., Rickettsia sp., Pasteurella sp., Proteus sp., Pseudomonas sp., Salmonella sp., Serratia sp., Shigella sp., Staphylococcus sp., Streptococcus sp., Treponema sp., Vibrio sp., Yersinia sp., or a combination thereof.

The parasite can be a pathogenic parasite, examples of which include Acanthamoeba spp., Balamuthia spp., Babesia sp., Balantidium coli, Blastocystic sp., Cryptospiridium sp., Cyclospora cayetanensis, Entamoeba histolytica, Giardia lamblia, Isospora bello, Leishmania sp., Naegleria foweri, Plasmodium sp., Rhinosporidium seeberi, Sarcocystis sp., Toxoplasma gondii, Trichomonas sp., Trypanosoma sp., or a combination thereof.

The fungus can be, for example, Aspergillus sp, Blastomyces sp, Candida sp, Coccidiodes sp, Crytococcus sp, Epidermophyton sp, Histoplasma sp, Malassezia sp, Microsporum sp, Mucor sp, Paracoccidioides sp, Pityriasis sp, Pneumocystis sp, Rhizopus sp, Trichophytan sp, or a combination thereof.

The cancer antigen can be, for example, from an animal cancer cell or a human cancer cell, such as baculoviral inhibitor of apoptosis repeat-containing 5 (BIRC5 or survivin); cancer/testis antigen 1 (NY-ESO-1); carcinoembryonic antigen (CEA); human epidermal growth factor receptor 2 (HER2/neu or ERBB2); melanoma antigen recognized by T cells 1 (MART-1); melanoma-associated antigen (MAGE) family; mucin 1, cell surface associated (MUC1); mucin 2, cell surface associated (MUC2); premelanosome protein (PMEL or gp100); programmed death-ligand 1 (PD-L1); prostate-specific antigen (PSA); synovial sarcoma, X breakpoint (SSX) family, including the translocations of these genes with the synaptotagmin (SYT) gene; tumor protein P53; tyrosinase; up-regulated in lung cancer 10 (URLC10); vascular endothelial growth factor receptor 1 (VEGFR1); vascular endothelial growth factor receptor 2 (VEGFR 2); Wilms' tumor protein (WT1); or a combination thereof.

Also disclosed is a method of expressing a heterologous protein in a host cell, the method comprising contacting a host cell with the HSV gene expression vector disclosed herein and measuring expression of the heterologous protein in the host cell. In an aspect, the method comprises contacting a host cell with the HSV gene expression vector disclosed herein and measuring expression of a plurality of heterologous proteins in the host cell. The contacting comprises transfecting and/or infecting the host cell with the HSV gene expression vector.

The present disclosure provides a recombination system for high-throughput cloning comprising first and second recombination partners. The first recombination partner comprises a genetically modified HSV-2 having a genome comprising a complete deletion of glycoprotein G-encoding gene, glycoprotein J-encoding gene, glycoprotein D-encoding gene, and glycoprotein I-encoding gene, and a first recombination sequence; and the second recombination partner comprises a heterologous nucleic acid encoding a second recombination sequence, and an expression cassette, wherein the second recombination sequence comprises an attP sequence from bacteriophage Bxb1, and the expression cassette comprises in operable communication a promoter and at least one gene encoding a heterologous protein.

In an aspect, the first recombination partner is the Δg(GJDI)-2::attB disclosed herein. In an aspect, the second recombination partner is the heterologous nucleic acid including the attP sequence and the expression cassette disclosed herein.

In an aspect, the second recombination sequence is upstream or downstream of the expression cassette.

In an aspect, the expression cassette comprises in operable communication the promoter, the at least one gene encoding the heterologous protein, and a polyadenylation signal.

Also disclosed herein is a method of expressing a heterologous protein in a host cell, the method comprising: providing the recombination system of the present disclosure comprising the first recombination partner and the second recombination partner; contacting the first recombination partner and the second recombination partner in the presence of a bacteriophage Bxb1 serine integrase and under conditions in which sequence-specific recombination occurs between the first recombination sequence and the second recombination sequence to provide a recombined HSV-2 genome; transfecting a complementing cell with the recombined HSV-2 genome; recovering an HSV virus or virion from the transfected complementing cell so as to obtain an HSV gene expression vector directed against the heterologous protein; and infecting the host cell with the HSV gene expression vector.

In an aspect, the method further comprises measuring expression of the heterologous protein in the host cell. Expression can be measured using methods known to those of skill in the art. Such methods include, but are not limited to, ELISA, Western blot, immunofluorescence, flow cytometry, or a combination thereof.

In an aspect, the second recombination partner comprises a heterologous nucleic acid comprising an expression cassette comprising a plurality of genes each encoding a different heterologous protein and in operable communication with the promoter.

In an aspect, the HSV vector is an HSV-2 vector which comprises HSV-1 glycoprotein D in the viral envelope.

In an aspect, the complementing cell expresses HSV-1 glycoproteins G, J D, and I and phenotypically complements the genetically modified HSV-2 gene expression vector. In an aspect, the HSV-2 gene expression vector comprises HSV-1 glycoproteins G, J D, and I in the viral envelope.

In an aspect, in non-complementing cells, the HSV-2 vaccine vector is a single cycle infectious virus.

In an aspect, a molar ratio of the genome of the genetically modified herpes simplex virus-2 to the heterologous nucleic acid is 1:1, 1:2, 1:3, 1:1 to 3:1, or equimolar.

In an aspect, an amount of Bxb1 serine integrase in the contacting is 0.01 picomole (pmol) to 5 pmol, or 0.2 pmol to 3 pmol, or 1 pmol to 2 pmol. In an aspect, the amount of Bxb1 serine integrase is 2 pmol.

Disclosed herein is a composition comprising the HSV-2 vectors described herein. Also provided is a vaccine composition comprising the HSV-2 vectors described herein. A pharmaceutical composition is also provided, the pharmaceutical composition comprising the HSV-2 vectors described herein, and a pharmaceutically acceptable carrier. Pharmaceutically acceptable carriers are well-known in the art.

In an aspect, the composition, vaccine composition or pharmaceutical composition is formulated so that it is suitable for administration to a subject. In an aspect, the subject is a mammalian subject. Administration can be auricular, buccal, conjunctival, cutaneous, subcutaneous, endocervical, endosinusial, endotracheal, enteral, epidural, via hemodialysis, interstitial, intrabdominal, intraamniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronary, intradermal, intradiscal, intraductal, intraepidermal, intraesophageal, intragastric, intravaginal, intragingival, intraileal, intraluminal, intralesional, intralymphatic, intramedullary, intrameningeal, intramuscular, intraocular, intraovarian, intraepicardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intraventricular, intravesical, intravitreal, laryngeal, nasal, nasogastric, ophthalmic, oral, oropharyngeal, parenteral, percutaneous, periarticular, peridural, rectal, inhalational, retrobulbar, subarachnoid, subconjuctival, sublingual, submucosal, topical, transdermal, transmucosal, transplacental, transtracheal, ureteral, urethral, or vaginal. A combination comprising at least one of the foregoing routes of administration can also be used.

Disclosed herein also are methods of eliciting and/or enhancing an immune response to at least one heterologous antigen in a subject. In an aspect, the method comprises administering the HSV-2 virus, virion or vaccine vector disclosed herein in an amount effective to elicit the immune response to the heterologous antigen in the subject. In an aspect, the HSV-2 vaccine vector comprises a plurality of genes each encoding a different heterologous antigen, and an immune response to at least one heterologous antigen is elicited or enhanced in the subject. The vaccine elicits an immune response comprising neutralizing antibodies and/or nonneutralizing antibodies able to activate effector immune functions in immune cells, such as antibody-dependent cellular cytotoxicity (ADCC). Without being bound to a theory, nonneutralizing antibodies stimulate effector cell mechanisms, including antibody-mediated phagocytosis and antibody-dependent cellular cytotoxicity (ADCC), both of which require activation of the Fcγ receptors (FcγRs). ADCC is initiated by immune cells, including killer cells and macrophages, when an Fc receptor is engaged by the Fc region of an antibody. This interaction activates a downstream signaling cascade that results in a cytotoxic response against the infected target cell. Among FcγRs, FcγRIV seems to have a central role in mediating ADCC. Specific isotypes of IgG antibodies are associated with binding and modulation of FcγR and subsequent ADCC activation, including the IgG1 and IgG3 subtypes in humans, as well as IgG2a and IgG2c subtypes in mice. ADCC-mediated antibodies have been demonstrated to protect against virus challenge by passive transfer of immune sera from vaccinated to nonvaccinated subjects.

Also provided is a method of eliciting and/or enhancing an immune response to a heterologous antigen in a subject, the method comprising administering a pharmaceutical composition or vaccine comprising the HSV-2 vaccine vector in an amount effective to elicit or enhance the immune response to the heterologous antigen in the subject.

The HSV vaccine vector disclosed herein may be formulated to include other medically useful drugs or biological agents. The viral vectors also may be administered in conjunction with the administration of other drugs or biological agents or vaccines useful for the disease or condition that is elicited by the organism from which the heterologous gene is derived, or for a different disease or condition elicited by a different organism.

Also provided is a method of inducing antibody dependent cell mediated cytotoxicity (ADCC) against an antigenic target in a subject comprising administering to the subject the HSV-2 vaccine vector in an amount effective to induce antibody dependent cell mediated cytotoxicity (ADCC) against an antigenic target. In one aspect, the antigenic target is a heterologous antigen expressed from a heterologous sequence in the HSV-2 vaccine vector. In an aspect, the HSV-2 vaccine vector comprises a plurality of genes each encoding a different heterologous antigen, and an immune response to at least one heterologous antigen is elicited or enhanced in the subject. In one aspect, the heterologous antigen is a HSV-1 antigen, a viral antigen, a bacterial antigen, a parasite antigen, a fungal antigen, a plant antigen, a cancer antigen, a protein from a mammalian cell, a protein from a non-mammalian cell, or a combination thereof.

A method is provided of eliciting an immune response in a first subject against an HSV-2 and/or HSV-1 infection, comprising effectuating passive transfer to the first subject of an amount of a product from a second subject immunized with HSV-2 vector described herein, wherein the product comprises serum antibodies induced thereby effective to elicit an immune response against an HSV-2, and/or HSV-1 infection in the first subject. Passive transfer or passive infusion of immune sera from a first subject administered an HSV vaccine vector with a heterologous antigen of the present invention to a second subject transfers protection to the second subject against HSV virus challenge and against challenge with the pathogen source of a heterologous antigen. Immune sera comprises serum containing antibodies, both monoclonal and polyclonal, obtained from a subject that has been subjected to exposure to an antigen either by vaccination or natural exposure. Immune sera is prepared from the blood of a subject after removing red blood cells.

In one aspect, the second subject a pregnant female immunized with a HSV-2 vector described herein and wherein the first subject is a fetus or neonate. In one aspect, the HSV-2 vector comprises a heterologous nucleic acid encoding a heterologous antigen from a pathogen wherein an immune response is elicited against the heterologous antigen sufficient to protect against infection with the pathogen. In one aspect, the pathogen is a virus, a bacteria, a fungus, a parasite. In one aspect, the heterologous antigen is a HSV-1 antigen, a viral antigen, a bacterial antigen, a parasite antigen, a fungal antigen, a plant antigen, a cancer antigen, a protein from a mammalian cell, a protein from a non-mammalian cell, or a combination thereof.

Also provided is a method of inhibiting a perinatal HSV-1 and/or HSV-2 infection in a neonate comprising administering to a female pregnant with a fetus which will become the neonate an amount of a HSV-2 virus vector described herein, effective to inhibit a perinatal HSV-1 and/or HSV-2 infection in a neonate. In one aspect, the HSV-2 vector comprises a heterologous nucleic acid encoding a heterologous antigen from a pathogen wherein an immune response is elicited against the heterologous antigen sufficient to protect against infection with the pathogen. In one aspect, the pathogen is a virus, a bacteria, a fungus, a parasite. In one aspect, the heterologous antigen is a HSV-1 antigen, a viral antigen, a bacterial antigen, a parasite antigen, a fungal antigen, a plant antigen, a cancer antigen, a protein from a mammalian cell, a protein from a non-mammalian cell, or a combination thereof.

Also provided is a method of inhibiting HSV-1 and/or HSV-2 viral dissemination from a mother to her neonate comprising administering to the mother an amount of an HSV-2 virus vector described herein, effective to inhibit HSV-1 and/or HSV-2 viral dissemination from a mother to her neonate. In one aspect, the HSV-2 vector comprises a heterologous nucleic acid encoding a heterologous antigen from a pathogen wherein an immune response is elicited against the heterologous antigen sufficient to protect against infection with the pathogen. In one aspect, the pathogen is a virus, a bacteria, a fungus, a parasite. In one aspect, the heterologous antigen is a HSV-1 antigen, a viral antigen, a bacterial antigen, a parasite antigen, a fungal antigen, a plant antigen, a cancer antigen, a protein from a mammalian cell, a protein from a non-mammalian cell, or a combination thereof.

This disclosure is further illustrated by the following examples, which are non-limiting.

### EXAMPLES

### Materials and Methods

Cells and HSV strains. Wild-type HSV strains, including HSV-1 B³x1.1 or HSV-2(4674), were grown on Vero cells (ATCC). These HSV strains were obtained from the Montefiore Clinical Virology laboratory (Petro, Weinrick, et al. 2016b). The recombinant ΔgD-2 and Δg(GJDI)-2 viruses were grown on the VD60 cell line, a complementing Vero cell line expressing glycoproteins G, J, D, and I from HSV-1 strain KOS (Ligas and Johnson 1988a). The construction of the ΔgD-2::RFP virus was described previously in Kaugars et al. 2021 (PNAS 118, e2110714118, doi.org/10.1073/pnas.2110714118).Generation of Δg(GJDI)-2 and Δg(GJDI)-2::attB. To construct the Δg(GJDI)-2, we deleted the US4, US5, US6 and US7 genes, encoding for the glycoproteins G, J, D, and I, respectively, by homologous recombination, as follows.

First, the HSV-2 strain G viral DNA was isolated (as described below), and a cosmid library was made by partially digesting the HSV-2 genomic DNA with BamHI and cloning of the specific fragment containing the US region into the pYUB328 cosmid (Balasubramanian et al., 1996 J. Bacteriol. 178 (1). doi: 10.1128/jb.178.1.273-279.1996) by lambda packaging, resulting in pYUB2156. The lambda packaging is a high efficiency transduction system to package methylated and unmethylated linear DNA containing lambda bacteriophage cohesive (cos) sites. The packaging extract contains lambda capsid proteins expressed in restriction-free E. coli K-12 strains and is capable of in vitro packaging DNA of 38-kb to 52-kb in size. The cosmid pYUB328 was digested with NheI and then dephosphorylated with rSAP (NEB). The rSAP was inactivated at 65° C. for 5 minutes. The linearized plasmid was then digested with BclI to liberate the two fragments. The HSV-2 genomic DNA was partially digested with BamHI to produce fragments between 40 and 50-kB in length. The pYUB328 fragments were ligated with partially digested HSV-2 DNA to produce concatemers of DNA. 10 μl of the ligated mixture was then packaged with Maxplax extract (Lucigen) and then transduced in competent HB101 cells. Subsequent colonies were selected by Sanger sequencing to isolate the clones.

Second, the selection marker, kan gene (flanked by SwaI sites), was PCR amplified from the pMV306 plasmid (MedImmune™, Gaithersburg, MD) using primers that contained homologous arms flanking the upstream region of US4 gene, gG FW primer (5′-AGACGCGGC CCTCGGGCTTTGGTGTTTTTGGCACCTTGCCGCCCGGCGTCATTTAAATCCCAAGG A CACTGAGTCCTAAAG-3′; SEQ ID NO: 9), and the downstream region of the US7 gene, gI RV primer (5′-GGGGGGGAAATAACCACGATGGGGGCGGTGGGGCGGGCCTGCCGAACG GCCATTTAAATAAGAAGGTGTTGCTGACTCATACC-3′; SEQ ID NO: 10).

Third, the resulting PCR product was used as a recombinogenic substrate together with the cosmid pYUB2156 to precisely delete the US4-US7 genes in E. coli (strain DY331: W3110 ΔlacU169 gal490 μglΔ8 [λ cI857 Δ (cro-bioA)] Δ (srlA-recA) 301::Tn10), resulting in pBRL950. The pBRL950 cosmid was then digested with SwaI to remove the kan gene and re-ligated, resulting in pBRL951. The pBRL951 plasmid was linearized with PacI and co-transfected with the ΔgD-2::RFP viral DNA into the complementing cell line VD60 (Ligas M. W., et al., Journal of Virology. 1988; 62(5):1486-94) using the Ingenio® Electroporation Kit (Mirus Bio), following the manufacturer's protocol. The VD60 cell line is a Vero cell line that complements both ΔgD-2 and Δg(GJDI)-2 viruses with the expression of the US4-US7 genes from KOS strain of HSV-1. The ΔgD-2::RFP is a HSV-2 in which a gene encoding RFP fused to the promoter of the Elongation Factor 1α (EF1α) gene replaces the Us6 gene of HSV-2, and is thus a ΔgD-2 virus expressing red fluorescent protein (RFP)(tdTomato). Recombinant plaques were selected that did not express red fluorescent protein and isolated plaques were submitted to three rounds of plaque purification. The final recombinant Δg(GJDI)-2 virus isolate was confirmed by Sanger sequencing.

Generation of Δg(GJDI)-2::attB viral vector: The 38-bp attB sequence of mycobacteriophage Bxb1 (5′-GGCTTGTCG ACGACGGCGGTCTCCGTCGTCAGGATCAT-3′, SEQ ID NO:1) was PCR amplified from plasmid G194A_AttB_sGFP (Matreyek et al., 2017, Nucleic Acid Res. 45 (11) e102. doi: 10.1093/nar/gkx183) with the primers G194_2904_attB-F (5′-GCGCGCGCTTATAACTCG AGCCGGCTTGTCGACG-3′; SEQ ID NO:11) and G194_2949_attB-R (5′-GCGCGCGCT TATAAATGATCCTGACGACGGAGAC-3′; SEQ ID NO:12) and digested with PsiL. The digested attB amplified product was cloned in the SwaI site of the cosmid pBRL951, between the US3 and US8 homologous arms, resulting in the cosmid pBRL962.

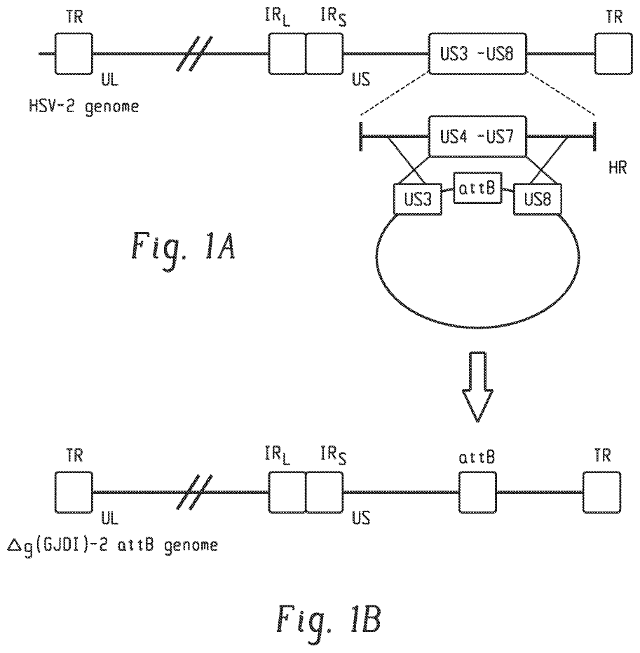

To generate the Δg(GJDI)-2::attB, pBRL962 was digested with PacI and co-transfected with ΔgD-2::RFP viral DNA into VD60 cells by Effectene reagent (Qiagen®) following the manufacturer's protocol. Non-fluorescent viral plaques were selected and submitted to three rounds of plaque purification, and the new recombinant Δg(GJDI)-2::attB (FIG. 1B) was confirmed by PCR amplification with Q5 Hot Start High Fidelity DNA polymerase (NEB, USA) with primers G194_2904_attB-F and G194_2949_attB-R (described above).

Immunofluorescence analysis: Briefly, VD60 cells were seeded in 24-well plates at 7×10⁴ cells/well. On the following day, growth media was replaced by DMEM with 2% FBS and cells were transfected with either the pMAE::dnaK or pMAE::groEL-2, pMAE::cut4, pMAE::cut7, pMAE::pstS1, pMAE1ppx (1 ug) using Lipofectamine® 3000 (as described in manufaturer's protocol, Invitrogen™) in OptiMEM and incubated at 37° C., 5% CO₂ for 48 h. Cells were washed with PBS, fixed with 4% paraformaldehyde in PBS for 10 minutes at room temperature, and treated with 0.1 M glycine for 5 minutes. Cells were permeabilized with 0.2% Triton X-100 in PBS for 15 minutes and blocked with 5% BSA in PBS for 1 hour at room temperature. Transfected cells were stained using primary antibodies specific against DnaK (NR-13609, ATCC), GroEL-2 (NR-13657, ATCC), or anti-His tag (Abcam, cat #ab18184) at a dilution of 1:500 for 16 h at 4° C. Following washing, cells were probed with a goat anti-mouse secondary antibody conjugated to a fluorescent green dye, Alexa Fluor™ Plus 488 (Thermo Fisher Scientific™, A32723), at a dilution of 1:1,000 for 1 h at room temperature. Images were captured using the EVOS 5000 Imaging System (Invitrogen™).

Virus production and viral DNA isolation. The Δg(GJDI)-2 and/or Δg(GJDI)-2::attB virus was grown in the complementing VD60 cell line expressing the gene for HSV-1 gG, gJ, gD, and gI (Ligas M. W., et al., Journal of Virology. 1988; 62(5):1486-94) to provide infectious, single cycle virus. The complementing VD60 cells were seeded at 1×10⁷ cells per 150 mm-dish in DMEM supplemented with 2% FBS. The next day, cells were infected with the respective viruses at an MOI of 0.01 plaque-forming units (PFU)/cell and cytopathic effect was followed by 3 days post-infection. Infected cells were scraped into the medium and centrifuged at 2,000 rpm for 10 minutes at 4° C. The cell pellet was resuspended in PBS and submitted to 3 cycles of freeze and thaw. The supernatant containing the released virions was clarified at 2,000 rpm for 10 minutes at 4° C. Virions were purified by a sucrose gradient (60%, 30%, and 10%) at 28,000 rpm for 2 hours at 4° C., followed by a washing and precipitation step at 22,000 rpm for 1 hour at 4° C. The virion pellet was resuspended in 200 μl of Hank's balanced salt solution (HBSS) overnight at 4° C. Purified virions were treated with 1,000 units (U) Benzonase for 4 hours at 37° C., followed by an inactivation step with 1 mM EDTA, for 15 minutes at 37° C. Viral DNA was released from the viral particle by a proteinase K treatment (1 mg proteinase K, 0.1% SDS in TNE buffer [0.1 M NaCl, 50 mM Tris-HCl pH 7.5, 10 mM EDTA]) for 1 hour at 37° C. Viral DNA was extracted by phenol:chloroform:isoamyl alcohol (25:24:1, pH 8.0), using MaxTract tubes (Qiagen®), followed by 90% Ethanol (3V): 3M Na acetate (0.1V) precipitation, and quantification by QUBIT™ (Invitrogen™)

Mouse vaccination, antibody analysis, and HSV-2 challenge: Ten female C57BL/6J mice from 4-5 weeks old were immunized intramuscularly in the hind leg following a prime-boost regimen three weeks apart. To evaluate protection conferred by the Δg(GJDI)-2 vector, mice were inoculated with 5×10⁶ plaque forming units (PFU) of Δg(GJDI)-2, 5×10⁶ PFU of ΔgD-2::RFP as a positive control, or VD60 cell lysate as a negative control. In addition, to test the generation of DENV-specific sera, mice were immunized with 5×10⁶ PFU Δg(GJDI)-2::DENV NS1 or the negative controls 5×10⁶ PFU Δg(GJDI)-2::attB or VD60 cell lysate. Blood samples were obtained at week 6.

The mice were then boosted with the same immunization 3 weeks later. The mice were then bled retro-orbitally at 6 weeks post-prime. For the HSV challenge experiments, mice were challenged two days after the bleed at week 6 with either 100xLD₅₀ HSV-1 B³×1.1 or 10×LD₉₀ HSV-2(4674) (already described above in the first section), according to a previously published protocol (Petro, Gonzilez, et al. 2015; Petro, Weinrick, et al. 2016a). One day prior to the challenge, the mice were depilated on the back leg to expose the skin. On the day of the challenge, the skin was abraded to remove the outermost layer of the skin, and the HSV virus was applied to the abraded area via pipette. Then, mice were monitored daily for two weeks for weight loss, epithelial disease, and neurological disease, scored as previously described (Petro, Christopher D., et al., JCI insight 1.12 (2016)). Mice were sacrificed if paralysis developed.

Enzyme-linked immunosorbent assay (ELISA). The sera from each of the groups were analyzed using enzyme-linked immunosorbent assay (ELISA) with HSV-2 virus lysate as a target, as previously described (Petro, Christopher D., et al JCI insight 1.12 (2016); Kaugars et al., 2021). Briefly, Maxisorp Immuno plates (Fisher Scientific™, Cat #12565136) were coated with lysates from Vero cells infected with HSV-2 (4674) of MOI 1 PFU/cell for 24 hours at 50 μg/mL or coated with 0.5 ug/ml recombinant NS1 protein (The Native Antigen Company, Cat #DENV2-NS1-100) Coated plates were incubated at 4° C. overnight and treated with 1% paraformaldehyde for 15 minutes. Plates were washed twice with sterile PBS and blocked with 5% non-fat dry milk solution in PBS to be incubated overnight at 4° C. Plates were then washed with PBS with 0.05% Tween-20. Serum was serially diluted 10-fold starting at dilution of 10², added to the plates, and incubated overnight at 4° C. Plates were washed with PBS with 0.05% Tween-20. Monoclonal secondary antibodies conjugated to biotin were added at a 1:500 dilution, and plates were incubated for 1.5 hours at 37° C. The secondary antibodies include Biotin Goat anti-mouse IgG (Biolegend, Cat #405303); Biotin Rat anti-mouse IgG1 (Biolegend, Cat #406604); Biotin Rat anti-mouse IgG2a (Biolegend, Cat #407104); Biotin Rat anti-mouse IgG2b (Biolegend, Cat #406704); and Biotin Rat anti-mouse IgG3 (Biolegend, Cat #406803) antibodies. Plates were washed with PBS with 0.05% Tween-20, and avidin-peroxidase (Sigma Aldrich, Cat #A3151) was added at a 1:1000 dilution. The plates were incubated at room temperature in the dark for 30 minutes. Plates were again washed with PBS with 0.05% Tween-20. TMB substrate reagent (BD Biosciences, Cat #555214) was then added to the plates and incubated for approximately 2-4 minutes. Then, 2 M H₂SO₄ was added to the plates, and absorbance was measured at 450-nm using a PerkinElmer VICTOR3™ Multilabel Plate Reader.

FcγRIV activation assay: To measure FcγRIV activation, the Promega Mouse FcγRIV ADCC Bioassay was used. The target Vero cells were seeded at 12,500 cells/well on white, flat-bottomed 96-well plates in Dulbecco's Modified Eagle Medium (DMEM) with 10% FBS and incubated at 37° C., 5% CO₂. After 6-8 hours, the media was removed, and cells were infected with the HSV-2 4674 virus at an MOI of 3 PFU/cell in serum-free DMEM and incubated for 1 hour at 37° C., 5% CO₂. Then, DMEM with 10% FBS was added, and the plates were left to incubate overnight at 37° C., 5% CO₂. The next day, serum from vaccinated mice was diluted 1:5 and heat-inactivated at 56° C. for 30 minutes. Serum was then diluted again and added in triplicate. The manufacturer's protocol was followed at this point, and individual sera were tested in triplicate. In general, the virus-containing media was taken off and the dilutions of sera were added with the FcγRIV-expressing Jurkat T cells from the kit. The plates were incubated at 37° C., 5% CO₂ for 6 hours. Then, the luciferin-containing reagent was added, and the plates were analyzed for luminescence on a Biotek Synergy H1 Hybrid Multi-Mode Reader. Fold FcγRIV induction was calculated as: (Relative light units [RLU] of individual sample with effector and target cells—RLU of background)/(RLU of activation of effector cells by target cells without antibody—RLU of background).

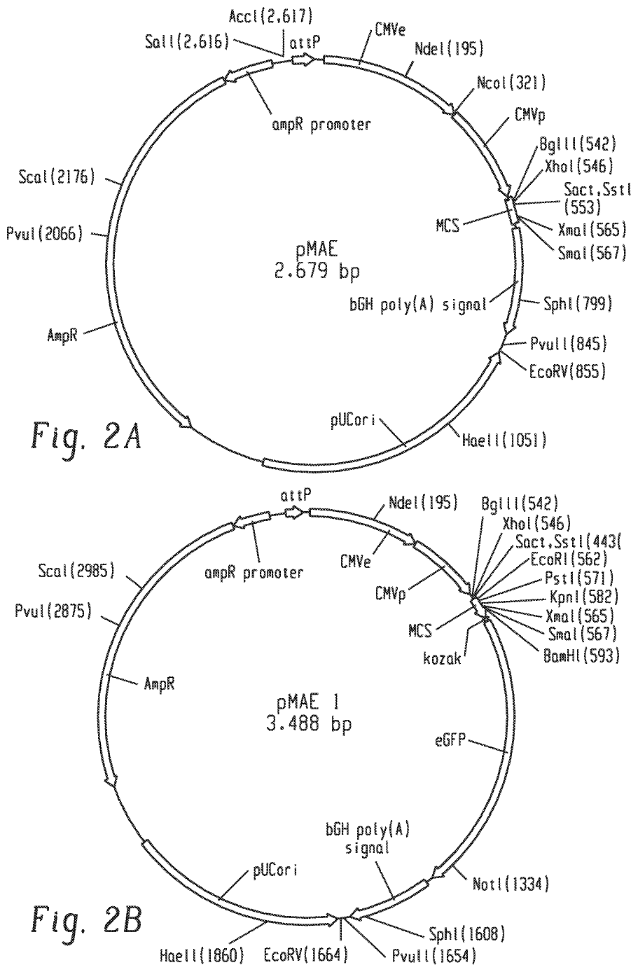

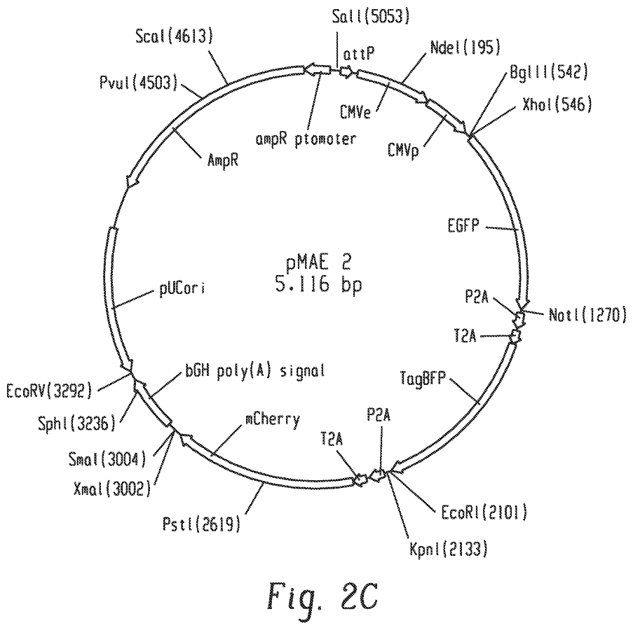

Construction of attP-containing plasmids for Bxb1 integrase-mediated recombination: One of the attP-shuttle plasmids was designed as a minimal plasmid, designated pMAE, to contain the Bxb1 attP sequence (5′-GGT TTG TCT GGT CAA CCA CCG CGG TCT CAG TGG TGT ACG GTA CAA ACC-′3′ (SEQ ID NO:2)) upstream of the CMV promoter and a multiple cloning site (MCS). A map of pMAE is illustrated in FIG. 2A. As a proof-of-concept, the reporter gene encoding for the green fluorescent protein (GFP) was inserted into the MCS of pMAE. Briefly, pcDNA3-GFP (AddGene, plasmid #74165) was digested with the restriction enzymes XhoI and PmeI, and the fragment containing the GFP gene sequence was gel purified and direct ligated (T4 Ligase, NEB) into the linearized pMAE, digested with XhoI and PmeI restriction enzymes, thereby generating pMAE1 (FIG. 2B). To evaluate the insertion capacity into the viral vector genome, an attP-shuttle plasmid containing a larger cassette was constructed. For that, the TagBFP-P2AT2A-EGFP-NLS-P2AT2A-mCherry cassette from the plasmid pcDNA5-MTS-TagBFP-P2AT2A-EGFP-NLS-P2AT2A-mCherry (AddGene, plasmid #87829) was digested with restriction enzymes BglII and SmaI, Klenow treated and ligated into the multiple cloning site of pMAE digested with SmaI by HiFi Assembly (NEB), resulting in pMAE2 (FIG. 2C). Following transformation into bacteria strain DH100 and plasmid DNA isolation, the pMAE1 and pMAE2 constructs were verified using Sanger sequencing.

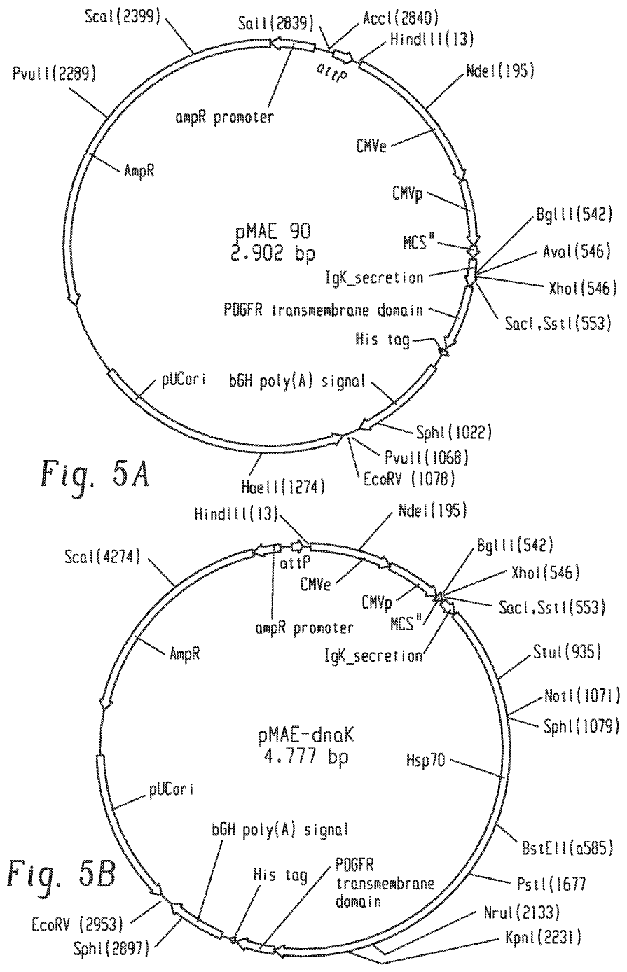

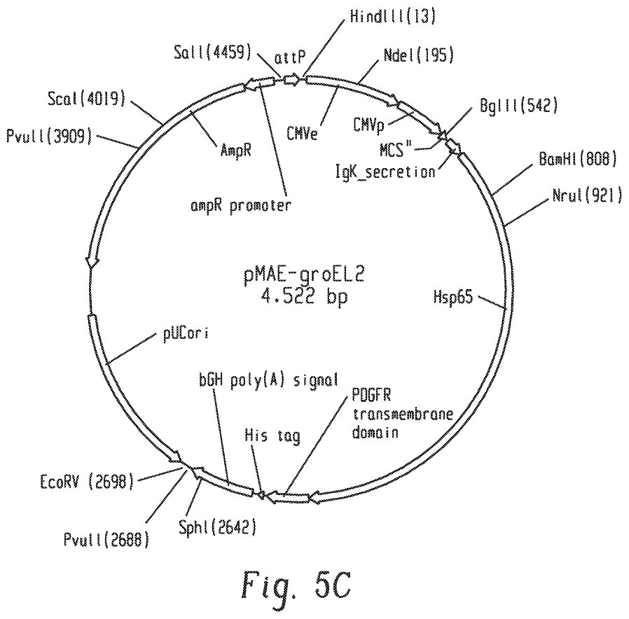

The pDisplay vector plasmid (Invitrogen™) contains a platelet-derived growth factor receptor (PDGFR) transmembrane domain and an IgK leader sequence to enhance expression and ensure that the gene of interest will be expressed on the cell surface. In order to enhance the expression of vaccine antigens on the surface of the infected cell, the expression elements from the pDisplay vector were digested with restriction enzymes SacI and HaeIII and inserted into the same restriction sites (SacI and SmaI) of the pMAE vector by direct ligation, resulting in pMAE90 (FIG. 5A). As a proof-of concept, we constructed shuttle plasmids encoding the dnaK gene (locus Rv0350, SEQ ID NO:30, amino acid sequence SEQ ID NO:31) or the groEL-2 gene (locus Rv0440, SEQ ID NO:32, amino acid sequence SEQ ID NO:33), cut4 (locus Rv2542, SEQ ID NO: 40, amino acid sequence SEQ ID NO:41), cut7 (locus Rv1984c, SEQ ID NO: 38, amino acid sequence SEQ ID NO: 39), lppX (locus Rv2945, SEQ ID NO: 42, amino acid sequence SEQ ID NO: 43), pstS1 (locus Rv0934c, SEQ ID NO:44, amino acid sequence SEQ ID NO: 45) Mycobacterium tuberculosis (strain H37Rv) genes. The M. tuberculosis transgenes were fused at the 5′-terminus to the IgK leader sequence and at the 3′-terminus to the PDGFR transmembrane domain sequence (primers HFVecp91_Hsp70, HFInspHsp65_p97, Table 1), by HiFi Assembly (NEB), according to manufacturer's protocol and using specific primers (Table 1), resulting in pMAE::DnaK (FIG. 5B) and pMAE::GroEL-2 (FIG. 5C), pMAE::cut4, pMAE::cut7, pMAE::1ppx, pMAE::pstS1 (data not shown).

TABLE 1
PRIMERS

| Primer name | Sequence |
|---|---|
| gG FW | AGA CGC GGC CCT CGG GCT TTG GTG TTT TTG GCA CCT |
| | TGC CGC CCG GOG TCA TTT AAA TCC CAA GGA CAC TGA |
| | GTC CTA AAG (SEQ ID NO: 9) |
| gI RV | GGG GGG GAA ATA ACC ACG ATG GGG GCG GTG GGG |
| | CGG GCC TGC CGA ACG GCC ATT TAA ATA AGA AGG TGT |
| | TGC TGA CTC ATA CC (SEQ ID NO: 10) |
| G194_2904_attB-F | GCG CGC GCT TAT AAC TCG AGC CGG CTT GTC GACG |
| | (SEQ ID NO: 11) |
| G194_2949_attB-R | GCG CGC GCT TAT AAA TGA TCC TGA CGA CGG AGAC |
| | (SEQ ID NO: 12) |
| US3_FW | ACT ACC CTC ATC GGG TAA TC (SEQ ID NO: 17) |
| US8_RV | AGG TTA CCC GTT TCC AGG AC (SEQ ID NO: 18) |
| Amp_D2gen_Mv5pD_F | ACT GCT AGA CCT GCA GGT CAG TGG TGG TGG TGG |
| | TGG TGT TCC TCA CGT GGC TTC T (SEQ ID NO: 19) |
| Amp_D2gen_pv5pD_R | ACT GCT AGA GTT TAA ACT CAG TGG TGG TGG TGG TGG |
| | TGT TCC TCA CGT GGC TTC T (SEQ ID NO: 20) |
| HFVecp91_Hsp70_R | GACCGCACGAGCCATGTCACCAGTGGAACCT (SEQ ID |
| | NO: 21) |
| HFVecp91_Hsp70_F | ACGGCCGGGAGGCCAAGGCTGTGGGCCAGGA (SEQ ID |
| | NO: 22) |
| HFInsHsp70_p91_F | AGGTTCCACTGGTGACATGGCTCGTGCGGTC (SEQ ID |
| | NO: 23) |
| HFInsHsp70_p91_R | CGTGTCCTGGCCCACAGCCTTGGCCTCCCGGCC (SEQ ID |
| | NO: 24) |
| HFInspHsp65_p97_F | TTCCAGGTTCCACTGGTGACATGGCCAAGACAATTGCGTAC |
| | (SEQ ID NO: 25) |
| HFInspHsp65_p97_R | GTGTCCTGGCCCACAGCGAAATCCATGCCACCCATGTC |
| | (SEQ ID NO: 26) |
| HFVecpHsp65_p97_F | GTACGCAATTGTCTTGGCCATGTCACCAGTGGAACCTGGAA |
| | (SEQ ID NO: 27) |
| HFVecpHsp65_p97_R | GACATGGGTGGCATGGATTTCGCTGTGGGCCAGGACAC |
| | (SEQ ID NO: 28) |
| Cut7_New_F | CCATATGATGTTCCAGATTATGCTATGACTCCACGCAGCC |
| | (SEQ ID NO: 46) |
| Cut7 New_R | GTGTCCTGGCCCACAGCATTTCCGGCGTGATCGAGCCTG |
| | (SEQ ID NO: 47) |
| pMAE83_Cut7_BB_F | AGCATAATCTGGAACATCATATGGATAGTC |
| | (SEQ ID NO: 48) |
| pMAE83_Cut7_BB_R | AATGCTGTGGGCCAGGACACGCAG |
| | (SEQ ID NO: 49) |
| Cut4_New_F | TATGATGTTCCAGATTATGCTGTGATCCCCGCCCGCAGCCG |
| | (SEQ ID NO: 50) |
| Cut4_New_R | GTCCTGGCCCACAGCATTGATCCTGCTCGCGACGAAACGC |
| | (SEQ ID NO: 51) |
| pMAE83_Cut4_BB_F | AATGCTGTGGGCCAGGACACGCAG |
| | (SEQ ID NO: 52) |
| pMAE83_Cut4_BB_R | AGCATAATCTGGAACATCATATGGATAGTCAC |
| | (SEQ ID NO: 53) |
| pMAE97F | gctgtgggccaggacacg |
| | (SEQ ID NO: 54) |
| pMAE97R | gtcaccagtggaacctggaac |
| | (SEQ ID NO: 55) |
| Rv2945cF | ctgggttccaggttccactggtgacatgaatgatggaaaacgggc |
| | (SEQ ID NO: 56) |
| Rv2945cR | cctcctgcgtgtcctggcccacagcgtcgacgttgacgggttc |
| | (SEQ ID NO: 57) |
| pMAE97F | gctgtgggccaggacacg |
| | (SEQ ID NO: 58) |
| pMAE97R | gtcaccagtggaacctggaac |
| | (SEQ ID NO: 59) |
| Rv0934cF | ctgggttccaggttccactggtgacatgaaaattcgtttgcatacgc |
| | (SEQ ID NO: 60) |
| Rv0934cR | cctcctgcgtgtcctggcccacagcgctggaaatcgtcgcgat |
| | (SEQ ID NO: 61) |

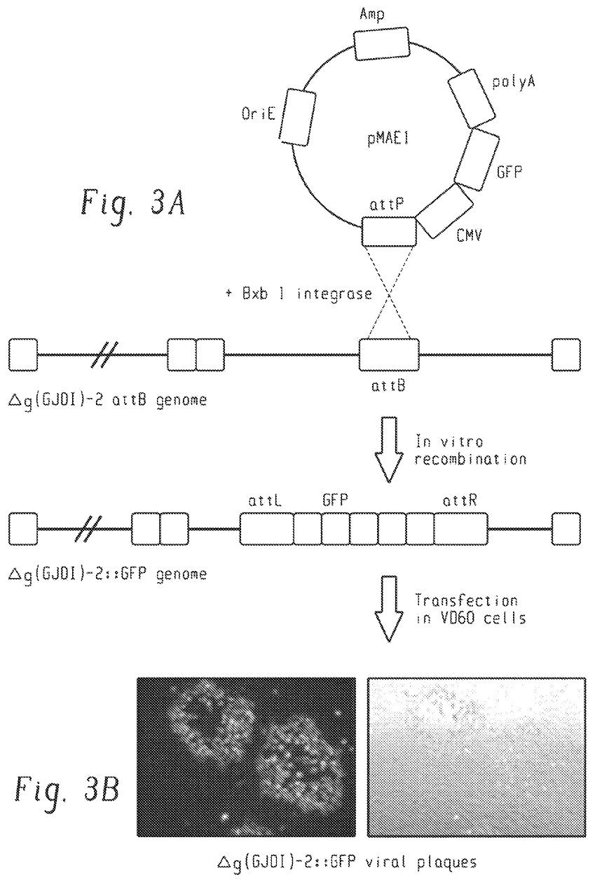

An additional plasmid was constructed to be compatible with the Bxb1 recombination system. pBKK700 contains the Bxb1 attP site, with an MCS with a CMV enhancer and EFla promoter and followed by Woodchuck Hepatitis Virus Posttranscriptional Regulatory Element (WPRE) and an SV40 polyadenylation signal sequence (polyA) (FIG. 3D). pBKK700 also contains a Kanamycin (Kan) resistance gene with its bacterial promoter and an mCherry reporter gene under a separate CMV promoter and enhancer.

Another shuttle plasmid was constructed for use in the Bxb1 integration system: pBKK745. The pBKK745 plasmid also contains the necessary attP site from the Bxb1 mycobacteriophage. The plasmid contains a gene expression cassette, consisting of a multiple cloning site (MCS.v4, which contains the restrictions sites for the restriction enzymes: SmaI, XmaI, AccI, Sal, BamHI, SphI, KpnI, NotI, PstI) preceded by a CMV promoter and enhancer and followed by a simian virus 40 polyadenylation signal (SV40 polyA). To generate the DENV recombinant virus, pBKK745 was digested with AscI and SbfI. We also amplified the NS1 gene from Dengue virus (DENV) serotype 2 (SEQ ID NO: 29), a kind gift from Dr. Peifang Sun at the Naval Medical Research Center. The plasmid with the original NS1 gene also contained a platelet-derived growth factor receptor (PDGFR) transmembrane domain and an IgK leader sequence (from the pDisplay vector, Invitrogen™ Addgene, Cat #V66020) to enhance expression and ensure that NS1 would be expressed on the cell surface. Additionally, the NS1 gene has His, hemagglutinin (HA), and c-myc tags. After amplifying the NS1 gene with PCR primers Amp_D2gen_Mv5pD_F (SEQ ID NO:19) and Amp_D2gen_pv5pD_R (SEQ ID NO:20), the amplicon was digested with AscI and SbfI. The digested pBKK745 was ligated to the digested NS1 gene to form pBKK840. The inclusion of the NS1 gene was verified using Sanger sequencing.

In vitro recombination assay: The in vitro integration recombination was performed as previously described (Ghosh P. et al., PLoS Biol. 2006; 4(6):e186). Recombination between the indicated attP site in pMAE and the attB site present in the linear recombinant Δg(GJDI)-2::attB genome was performed using different DNA molar ratios of shuttle plasmid and viral genome mixed in the Recombination Buffer (20 mM Tris-HCl [pH 7.5], 10 mM EDTA, 25 mM NaCl, 10 mM spermidine, 1 mM dithiothreitol (DTT). Purified Bxb1 integrase was diluted in the Enzyme Buffer (10 mM Tris-HCl [pH 7.5], 0.1 mM dithiothreitol, 1 mg/ml bovine serum albumin), and added to the reaction mix at different concentrations. Reaction was carried out at 37° C. for 2 hours or at 25° C. for 16 hours, followed by heat inactivation at 75° C. for 15 minutes. The resulting construct was transfected into complementing cells, and clarified virus suspension were harvested from the transfected cells.

Rescue of recombinant virus: The recombinant Δg(GJDI)-2::pMAE was rescued in the complementing cell line, VD60. Specifically, 2×10⁶ VD60 cells were seeded per well in a 6 well-plate in DMEM containing 2% FBS. On the following day, cells were transfected with the in vitro recombination reaction product at a 1:2:2 ratio of DNA: P3000: Lipofectamine® 3000 (according manufacturer's protocol). Cytopathic effect correspondent to herpesvirus viral plaques was monitored for the expression of fluorescent protein (GFP or RFP). Transfected cells were harvested 3 days after transfection and submitted to 3 cycles of freeze/thaw to release cell-attached virions. Supernatant containing virions was clarified at 2000 rpm for 10 min at 4° C., and virus stocks were aliquoted and stored at −80° C.

Plaque purification and virus production: Complementing VD60 cells were seeded on 6-well plates and infected the next day with clarified virus suspension harvested from the transfected cells. Specifically, a serial 10-fold dilution of the virus suspension was made in DMEM and added to the cells in duplicate; infection was allowed to occur for 1 hour at 37° C., 5% CO₂. The viral inoculum was removed, cells were washed with PBS and a semi-solid medium (M199 containing 0.5% low melting point agarose, 2% FBS) was added to the cells. At 3 days post infection, single HSV plaques expressing GFP were picked and passaged into new VD60 cells. Three cycles of plaque purification were performed. After the third plaque purification step, Δg(GJDI)-2::GFP was used to infect 1×10⁶ complementing VD60 cells seeded in 150-mm² dish. At 3 days post-infection, infected cells were harvested and clarified at 2,000 rpm for 10 minutes at 4° C., and the cell pellet was resuspended in PBS. The cell pellet was subjected to 3 rounds of freeze and thaw, and the supernatant containing virions was clarified at 2,000 rpm for 10 minutes at 4° C. Virus stocks were aliquoted and stored at −80° C.

PCR verification ofplasmid integration: Integration of the attP-shuttle plasmid containing the transgene expression cassette into the Δg(GJDI)-2::attB genome was confirmed by PCR using primers flanking the insertion region in the rHSV-2 genome. For that, DNA from the Δg(GJDI)-2 recombinants was extracted using the MiniAmp Virus Elute kit (Qiagen®), according to manufacturer's protocol. PCR reaction was carried out with the forward primer US3_FW (5′-ACT ACC CTC ATC GGG TAA TC-3′-SEQ ID NO:17) and the reverse primer US8_RV (5′-AGG TTA CCC GTT TCC AGG AC-3′-SEQ ID NO:18) using the LongAmp® Taq 2X Master Mix (NEB) at an annealing temperature of 60° C., according to manufacturer's protocol.

Protection of Δg(GJDI)-2 vaccine: Mice immunizations, serum collection, and HSV challenge: Four to five week-old C57BL/6 female mice were immunized intramuscularly in the hind leg following a prime-boost regimen three weeks apart. To evaluate protection conferred by the Δg(GJDI)-2 vaccine vector, mice were inoculated with 5×10⁶ plaque-forming units (PFU) Δg(GJDI)-2, 5×10⁶ PFU ΔgD-2::RFP (positive control), or VD60 cell lysate (negative control). In addition, to test the generation of DENV-specific sera, mice were immunized with 5×10⁶ PFU Δg(GJDI)-2::DENV NS1 or the negative controls 5×10⁶ PFU Δg(GJDI)-2::attB or VD60 cell lysate. Blood samples were obtained at week 6.

Statistical analysis: Analyses were performed using GraphPad® Prism version 9.2.0 software (GraphPad® Software Inc., San Diego, CA). A p value of 0.5 was deemed statistically significant. Two-way analysis of variance (ANOVA) was used to analyze neurological and epithelial disease, one-way ANOVA was used for ELISA and FcγRIV activation analysis, and the Gehan-Breslow-Wilcoxon test was used for survival curves. All data are shown as mean±SD.

### Example 1

Construction of Δg(Gjdi)-2 and Δg(Gjdi)-2::Attb Viral Vector

A recombinant HSV-2 strain, Δg(GJDI)-2, was constructed to provide a versatile gene expression vector with increased cloning capacity. To do so, the HSV-2 genes US4, US5, US6, and US7; which encode the glycoproteins G, J, D, and I, respectively, were deleted from HSV-2 by homologous recombination (FIGS. 1A-B). To generate Δg(GJDI)-2::attB strain, the attB site from M. smegmatis was inserted into the US4-US7-deleted region of Δg(GJDI)-2 genome by homologous recombination. Briefly, the pBRL951 cosmid containing the attB sequence cloned between the homologous sequences flanking the US4-US7 region was co-transfected with the ΔgD-2::RFP viral DNA in the VD60 complementing cell line as illustrated in FIGS. 1A. Recombinant HSV-2 viral plaques that did not express the red fluorescent protein (RFP) were plaque purified 3 times. Δg(GJDI)-2::attB virus was grown in complementing VD60 cells and insertion of the attB sequence was confirmed by Sanger sequencing.

### Example 2

Δg(GJDI)-2 is Immunogenic and Protective Against 10×Ld₉₀ HSV-2 Challenge

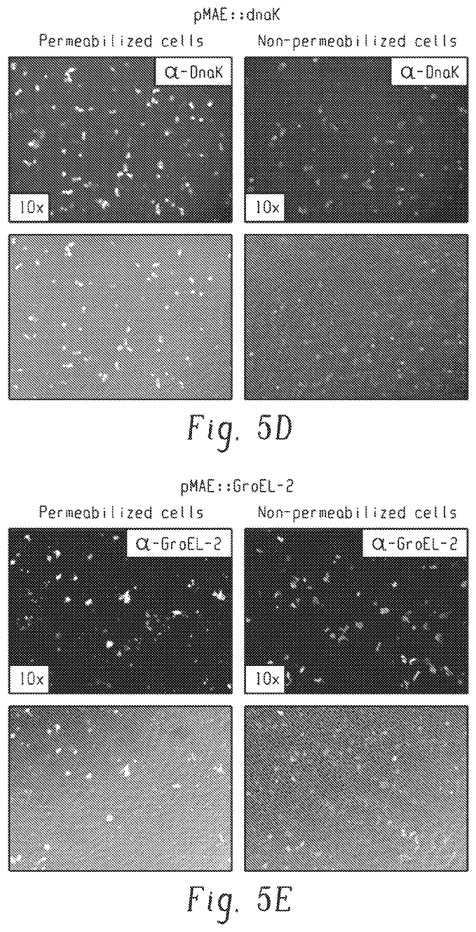

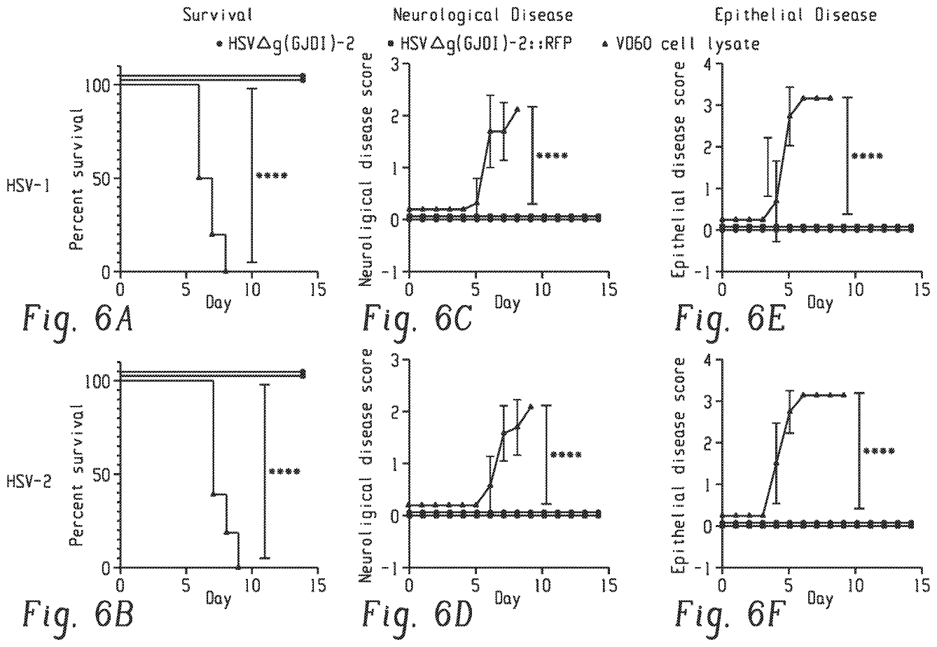

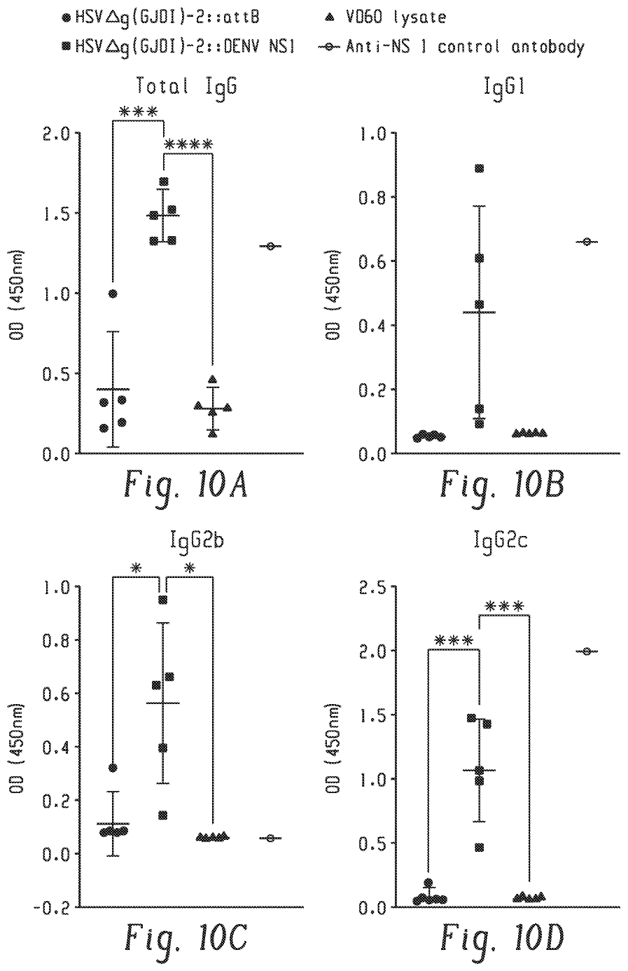

Female C57BL/6J mice from 4-5 weeks old were primed and boosted three weeks later, intramuscularly, with 5×10⁶ PFU of Δg(GJDI)-2, 5×10⁶ PFU of ΔgD-2::RFP as a positive control, or VD60 cell lysate as a negative control. The mice were then bled retro-orbitally at 6 weeks post-prime and challenged with either 100xLD₅₀ of HSV-1 (FIG. 6A) or 10xLD₉₀ of HSV-2 (FIG. 6B) HSV-2 via skin scarification. Mice were then assessed for epithelial and neurological disease daily for 2 weeks, and mice were sacrificed if paralysis developed. Mice vaccinated with either Δg(GJDI)-2 or ΔgD-2::RFP were protected against HSV-1 and HSV-2 (FIG. 6A, 6B), both neurological (FIG. 6C, 6D) and epithelial (FIG. 6E, 6F) disease. In both HSV-1 and HSV-2 challenges, mice given VD60 lysate developed severe epithelial disease, became paralyzed, and were sacrificed (p>0.0001, FIGS. 6A-F). The Δg(GJDI)-2 vaccine vector induces similarly high levels of protection as ΔgD-2::RFP against both HSV-1 and HSV-2 challenges.

### Example 3

Vaccination with Δg(GJDI)-2 Produces an IgG2c-Predominant Antibody Response Against HSV-2 that Strongly Activates FcγRIV

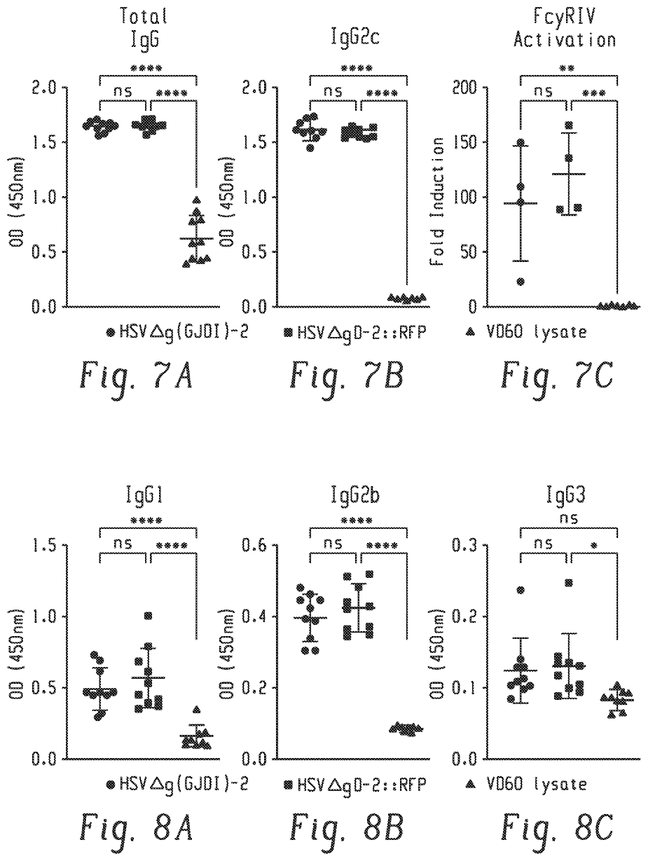

Serum was collected from all three vaccination groups (above) two days before challenge. Compared with mice mock-vaccinated with VD60 lysate, serum from the Δg(GJDI)-2-vaccinated mice had 2.6-fold higher levels of total IgG (p<0.0001, FIG. 7A), 3-fold IgG1 (p=0.0002, FIG. 8A), 4.6-fold IgG2b (p<0.0001, FIG. 8B), and 23.4-fold IgG2c (p<0.0001, FIG. 7B); however, there was no difference in IgG3 titers between these groups (FIG. 8C). Additionally, the sera from both Δg(GJDI)-2- and ΔgD-2::RFP-vaccinated mice had significantly higher FcγRIV activation than those vaccinated with VD60 lysate, 60.7- and 78-fold higher, respectively (p=0.016 and p=0.004, FIG. 7C). No significant differences in antibody titers or FcγRIV activation were found in the serum in mice vaccinated with either Δg(GJDI)-2 or ΔgD-2::RFP (FIG. 7 and FIG. 8). Consistent with high IgG2c titers and high FcγRIV activation, these results suggest that the in vivo mechanism of protection could be related to ΔDCC activity induction.

### Example 4

Construction of attP-Containing Shuttle Plasmids for Bxb1 Integrase-Mediated Recombination

A shuttle plasmid, responsible for transferring the transgene of interest into the Δg(GJDI)-2::attB genome, was designed to contain the Bxb1 phage attP site (FIG. 2A). The attP sequence was placed upstream of a CMV promoter, thus allowing the direct integration of the transgene expression cassette into the Δg(GJDI)-2::attB viral genome. Shuttle plasmids containing reporter genes were constructed by direct cloning of the genes encoding GFP (pMAE1) or BFP-GFP-mCherry (pMAE2) (FIGS. 2B, 2C).

In addition, shuttle plasmids containing the M. tuberculosis genes, DnaK, groEL-2, cut4, cut7, 1ppx, or pstS1 fused to the cell surface expression elements, IgK signal peptide and PDGFR transmembrane sequence, were constructed by HiFi assembly (NEB) using specific primers as shown in Table 1. To amplify the pMAE90 plasmid vector (FIG. 5A) (HFVecp91 Hsp70 R (SEQ ID NO:21) and HFVecp91_Hsp70_F (SEQ ID NO:22); HFVecpHsp65_p97_F (SEQ ID NO:25) and HFVecpHsp65_p97_R (SEQ ID NO:26)) and the insert genes dnaK (HFInsHsp70_p91_F (SEQ ID NO:23) and HFInsHsp70_p91_R (SEQ ID NO:24)), groEL-2 (HFInspHsp65_p97_F (SEQ ID NO:25) and HFInspHsp65_p97_R (SEQ ID NO:26)), resulting in pMAE::dnaK and pMAE::groEL-2, respectively (FIGS. 5B, 5C). Primers for amplifying insert genes cut4: pMAE83_Cut4_BB_F (SEQ ID NO:52) and pMAE83_Cut4_BB_R (SEQ ID NO:53); cut7: pMAE83_Cut7_BB_F (SEQ ID NO: 48) and pMAE83_Cut7_BB_R (SEQ ID NO:49); 1ppx: Rv2945cF (SEQ ID NO: 56) and Rv2945cR (SEQ ID NO: 57); pstS1: Rv0934cF (SEQ ID NO:60) and Rv0932cR (SEQ ID NO: 61) resulted in pMAE::Cut4, pMAE::Cut7, pMAE::1ppx, pMAE::PstS1, respectively (data not shown).

Expression of DnaK, GroEL-2, cut4, cut7, 1ppx, and pstS1 proteins in Vero cells transfected with the respective plasmids, pMAE::dnaK, pMAE::groEL-2, pMAE::cut4, pMAE::1ppx, and pMAE::pstS1 was confirmed by immunofluorescence analysis using antibodies specific to the inserted M. Tuberculosis proteins. Importantly, DnaK, GroEL-2, cut4, cut7, 1ppx, and pstS1 proteins were detected in the cytoplasm as well as on the surface of the transfected cells (FIGS. 5D, 2E, and data not shown).

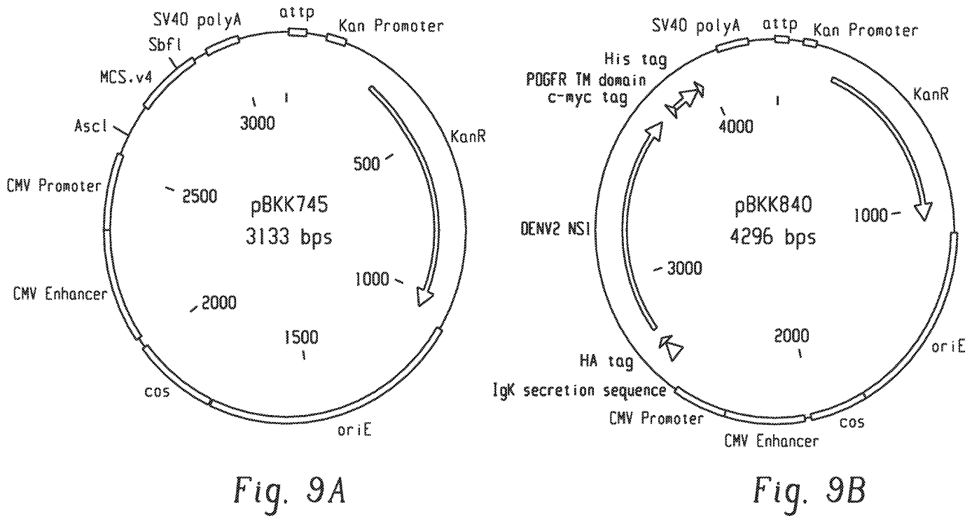

Another plasmid was constructed for use in the Bxb1 integration system: pBKK745 (FIG. 9A). The pBKK745 plasmid contains the necessary attP site from the Bxb1 mycobacteriophage (FIG. 9A). The plasmid also contains a gene expression cassette, consisting of a multiple cloning site preceded by a CMV promoter and enhancer and followed by a simian virus 40 polyadenylation signal (FIG. 9A).

### Example 5

Bxb1 Integrase Mediates Efficient and Stable Recombination Between attB and attP Recombination

To test if Bxb1 integrase-mediated recombination system could catalyze the recombination between the indicated attP site in pMAE1 and the attB site present in the Δg(GJDI)-2::attB genome (FIG. 3) to generate Δg(GJDI)-2::GFP, the in vitro reaction was optimized using different conditions. First, the most efficient Bxb1 integrase concentration was established. The Bxb1 integrase in concentrations of 0.02, 0.2, and 2 μmol, were tested using a fixed ratio of pMAE1 and Δg(GJDI)-2::attB viral DNA that allowed the highest recombination efficiency. The results are shown in Table 2.

TABLE 2
OPTIMIZATION OF BXB1 INTEGRASE ENZYME CONCENTRATION

| | # GFP + HSV | # GFP − HSV | Total # HSV | Recombination |
|---|---|---|---|---|
| Condition | plaques | plaques | plaques | efficiency (%) |
| Δg(GJDI)-2 attB (1):pMAE1 | 5 | — | 5 | 100 |
| (1) 2 h, 37° C. - Bxb1 Int (2 | | | | |
| pmol) | | | | |
| Δg(GJDI)-2 attB (1):pMAE1 | 7 | 2 | 9 | 78 |
| (1) 2 h, 37° C. - Bxb1 Int (0.2 | | | | |
| pmol) | | | | |
| Δg(GJDI)-2 attB (1):pMAE1 | 1 | 4 | 5 | 20 |
| (1) 2 h, 37° C. - Bxb1 Int (0.02 | | | | |
| pmol) | | | | |

Then, the molar ratio of viral (attB) DNA to plasmid (attP) DNA that allowed the highest recombination efficiency was tested. To do so, plasmid and viral DNA concentrations were measured by QUBIT (Invitrogen), and specific molar ratios of viral DNA to plasmid DNA of 1:1, 1:2, 2:1, 1:3, and 3:1, were tested (Table 3).

TABLE 3
RATIO OF Δg(GJDI)-2::ATTB VIRAL
DNA TO pMAE1 attP SHUTTLE PLASMID DNA.

| Ratio of | # GFP + | #GFP − | Total # | |
|---|---|---|---|---|
| Δg(GJDI)-2::attB | HSV | HSV | HSV | Recombination |
| DNA to pMAE1 DNA | plaques | plaques | plaques | efficiency (%) |
| 1:1 | 28 | 0 | 28 | 100 |
| 1:2 | 27 | 1 | 28 | 96 |
| 1:3 | 20 | 5 | 25 | 80 |

To rescue the recombinant Δg(GJDI)-2::GFP virus, the in vitro integration reaction product was transfected into complementing VD60 cells. The efficiency of Bxb1 integration-mediated recombination was assessed by expression of GFP in cells showing signs of cytopathic effect caused by HSV, specifically of viral plaques. For each condition, the number of GFP-positive and GFP-negative HSV viral plaques were counted as an average of triplicates, and recombination efficiency was calculated as the percentage of GFP-positive plaques. The results are shown in Table 4. The in vitro integration reaction was most efficient when carried out at 2 hours at 37° C. in Recombination Buffer (described in Methods), in the presence of 2 pmol purified Bxb1 integrase diluted in Enzyme Buffer (described in Methods), followed by heat inactivation at 75° C. for 15 minutes. Although the integration reaction mediated by Bxb1 integrase was shown to be highly efficient using the different ratios of plasmid and viral DNA, using equimolar viral and plasmid DNA leads to an increased generation of recombinant Δg(GJDI)-2::GFP virus.

TABLE 4: The in vitro Bxb1 integrase-mediated recombination reaction was carried out in the presence of different concentrations of Bxb1 integrase, using an equal molar ratio of Δg(GJDI)::attB viral and pMAE1 plasmid DNA for 2 hours at 37° C.

TABLE 4

| Bxb1 integrase | # GFP + | #GFP − | Total | Recombination |
|---|---|---|---|---|
| concentration | HSV | HSV | HSV | efficiency |
| (pmol) | plaques | plaques | plaques | (%) |
| 2 | 5 | 0 | 5 | 100 |
| 0.2 | 7 | 2 | 9 | 78 |
| 0.02 | 1 | 4 | 5 | 20 |

### Example 6

### Integration Analysis

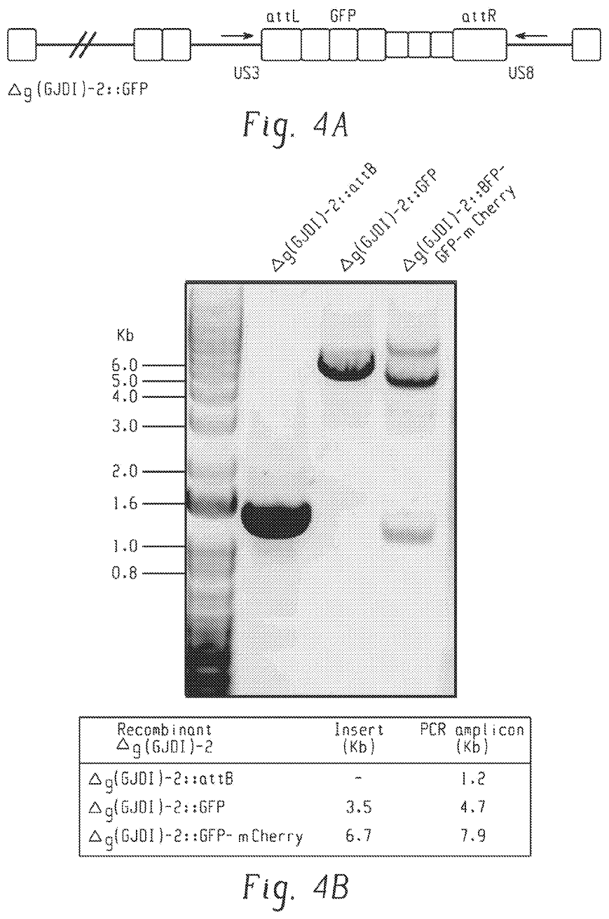

Following the Bxb1 integrase-mediated recombination reaction using the established conditions, the recombinant Δg(GJDI)-2::GFP virus was grown in the complementing VD60 cell line, and viral DNA was extracted. Integration of the attP-transgene expression cassette into the Δg(GJDI)-2::attB genome was confirmed by PCR amplification of the integrated attP transgene expression cassette using primers flanking the insertion region in the Δg(GJDI)-2 genome. (FIG. 4A).

To test the capacity of Bxb1 integrase to mediate the insertion of larger attP transgene cassettes into the Δg(GJDI)-2::attB genome, a shuttle plamid containing the attP site and multicistronic reporter genes encoding for BFP (blue fluorescent protein), GFP, and mCherry was constructed, generating pMAE2 (FIG. 2C). Integration analysis showed that up to 6 kb could be inserted into the Δg(GJDI)-2 genome and stably maintained over multiple passages (FIG. 4B).

### Example 7

Bxb1 Integrase-Mediated Recombination into Δg(GJDI)-2 Viral Genome is Stable

In order to evaluate the stability of the integrated attP transgene cassette into the Δg(GJDI)-2::attB genome, serial passages of the recombinant Δg(GJDI)-2::GFP virus were performed by infecting complementing VD60 cells. Viral DNA from Δg(GJDI)-2::GFP virus recovered from each viral passage was extracted and submitted to PCR amplification using primers flanking the US3 and US8 integration region (FIG. 4A). The integrated DNA from pMAE1 was stably maintained in the recombinant Δg(GJDI)-2 viral genome for at least 4 passages of Δg(GJDI)-2::GFP in VD60 cells.

### Example 8

### A Vaccine Created Using Bxb1 Integrase Mediated Recombination can Generate an Immune Response Against Dengue Virus NS1

As a proof-of-concept for the use of this system to create immunogenic vaccines, we used the pBKK745 plasmid (FIG. 9A) and cloned into it the NS1 gene from Dengue virus (DENV) serotype 2 to generate the pBKK840 plasmid (FIG. 9B). The sequence of the cloned, codon optimized sequence for eukaryotic expression DENV2 NS1 is

(SEQ ID NO: 29)
GTGCGGCAGCGGCATCTTCATCACCGACAATGTGCACACCTGGACCGAG
CAGTACAAGTTCCAGCCTGAGAGCCCTTCTAAGCTGGCCTCTGCCATCC
AGAAGGCCCAAGAAGAGGGCATCTGCGGCATCAGAAGCGTGACCAGACT
GGAAAACCTGATGTGGAAGCAGATCACCCCTGAGCTGAACCACATCCTG
GCCGAGAACGAAGTGAAGCTGACCATCATGACCGGCGACATCAAGGGCA
AGGCCACAGCCTACCGAGCTGAAGTACAGCTGGAAAACATGGGGCAAAG
CTCATGCAGGCCGGAAAAAGAAGCCTGCAAGATGCTGAGCACCGAGAGC
CACAACCAGACCTTTCTGATCGACGGCCCTGAGACAGCCGAGTGTCCCA
ATACCAACAGAGCCTGGAACAGCCTGGAAGTGGAAGATTACGGCTTCGG
CGTGTTCACCACCAACATCTGGCTGAAGCTGAAAGAGAAGCAGGACGCC
TTCTGCGACAGCAAGCTGATGTCTGCCGCCATCAAGGACAATAGAGCCG
TGCACGCCGACATGGGCTATTGGATCGAGAGCGCCCTGAACGACACCTG
GAAGATCGAGAAGGCCTCCTTCATCGAAGTCAAGAACTGCCACTGGCCT
AAGAGCCACACACTGTGGTCCAATGGCGTGCTGGAAAGCGAGATGATCA
TTCCCAAGAACCTGGCTGGCCCCGTGTCTCAGCACAACTACAGACCTGG
CTACCACACACAGATCGCCGGACCTTGGCACCTGGGCAAGCTGGAAATG
GACTTCGATTTCTGCGACGGCACCACCGTGGTGGTCACCGAGGATTGTG
GCAATAGAGGCCCTAGCCTGAGAACCACAACAGCCAGCGGAAAGCTGAT
CACCGAGTGCTGCTGCAGAAGCTGTACCCTGCCTCCTCTGAAATACAGA
AGCGAGGATGGCTGGTGGTACCGCATGGAAATCAGGCCCCTCAGAG

The pBKK840 plasmid was then combined with DNA from Δg(GJDI)-2::attB and the Bxb1 integrase to form Δg(GJDI)-2::DENV NS1. The insertion of the NS1 gene into Δg(GJDI)-2::DENV NS1 was verified using Sanger sequencing. Mice were primed with Δg(GJDI)-2::attB (empty vector), Δg(GJDI)-2::DENV NS1 or VD60 lysate and boosted after three weeks. After three additional weeks, mice were bled retro-orbitally. Serum levels of IgG directed against DENV2 NS1 was analysed by ELISA. As a positive control in the ELISA, an anti-NS1 antibody was used.

The mice vaccinated with Δg(GJDI)-2::DENV NS1 had 4.1- and 5.1-fold higher levels of total IgG antibodies relative to those receiving Δg(GJDI)-2::attB or VD60 lysate (p=0.0001 and p<0.0001, respectively, FIG. 10A). There were no significant differences between any of the groups in the levels of IgG1 antibodies (FIG. 10B). The positive control anti-NS1 antibody did seem to have some reactivity in the IgG1 isotype, despite being of the IgG2a isotype (FIG. 10B). The mice vaccinated with Δg(GJDI)-2::DENV NS1 had 5- and 10-fold higher levels of IgG2b antibodies relative to those receiving Δg(GJDI)-2::attB or VD60 lysate (p=0.0238 and p=0.0104, respectively, FIG. 10C). The mice receiving Δg(GJDI)-2::DENV NS1 had 13- and 19-fold higher IgG2c antibody levels relative to those vaccinated with Δg(GJDI)-2::attB or VD60 lysate (p=0.0002 and p=0.0002, respectively, FIG. 10D). Thus, the Δg(GJDI)-2::DENV NS1 vaccine had a significant anti-NS1 antibody response with high levels of total IgG, IgG2b, and IgG2c subtypes.

Described herein is an optimized HSV vector. Specifically, four genes from the HSV-2 genome were deleted to provide a system for efficient integration of genetic material into this virus. Despite the 6-kb deletion, the Δg(GJDI)-2 vaccine vector protects comparably to a single-deletion mutant, ΔgD-2, against lethal challenges from both HSV-1 and HSV-2. Δg(GJDI)-2 also elicits high levels of IgG2c antibodies that potently activate FcγRIV receptors. This vector can also be used in the Bxb1 mycobacteriophage integrase-mediated recombination system to generate HSV vector recombinants with stable transgene expression. A recombinant Δg(GJDI)-2 strain expressing a DENV protein, NS1, was immunogenic and elicited high levels of IgG2c antibodies, which are consistent with high levels of antibodies that mediate FcγRIV receptors activation.

Besides gD, all of the other three genes deleted from Δg(GJDI)-2 are considered nonessential for growth in vitro (Nguyen and Blaho. 2006. Apoptosis During Herpes Simplex Virus Infection.’ in, Advances in Virus Research (Elsevier); Johnson, D. et al. 1988. J Virol, 62: 1347-54; Daikoku, et al. 2013, Journal of Medical Virology, 85: 1818-28). gG (US4) has a largely unknown function but can bind to chemokines (Viejo-Borbolla, et al., PLoS Pathogens, 8: e1002497). gJ (US5) aids in the cell-to-cell spread of HSV-2 and the formation of syncytia (Liu, et al. 2018. Virology, 525: 83-95). In HSV-1, gJ can also block host cell apoptosis through the granzyme B or Fas pathways (Nguyen and Blaho 2006). gI (US7) associates with glycoprotein E (gE) to create a heterodimer mimicking an Fc receptor able to bind IgG (Johnson et al. 1988). In this capacity, gI is relevant to immune evasion by inhibiting the ability of IgG from the immune system to activate effector functions, such as ADCC (Lubinski, et al., 2011. Journal of Virology, 85: 3239-49). gI also is used in the cell-to-cell spread of the virus (Hilterbrand et al. 2019. PLOS Pathogens, 15: e1007660). Balan, et al. 1994 (J. Gen Virol. 75: 1245-58) completed single deletions in an HSV-1 backbone of either gG, gJ, or gI to analyze how the deletion of these genes would affect in vitro and in vivo phenotypes (Balan et al. 1994). Single deletions of gJ and gG did not produce any change in vitro or in vivo (Balan et al. 1994). However, deletion of the gI gene leads to viruses that produced smaller plaques in cell culture and an attenuated phenotype in vivo (Balan et al. 1994). Relative to the wild-type parental strain, mice challenged with the HSV strain deleted for gI quickly cleared the virus from the site of infection and developed very little detectable virus in nervous tissue (Balan et al. 1994). In a separate study, deletion of even a single nonessential glycoprotein (gC) drastically reduced the in vitro growth kinetics in a non-complementing cell line and the in vivo pathogenicity of the virus in a murine intracerebral challenge model (Schranz, et al., 1989. Virology 170:273-276).

Other viral vectors could greatly benefit from getting more cloning capacity. In the case of Δg(GJDI)-2, the literature predicted that deleting gI would severely attenuate the virus to the point that it cannot propagate well in vivo (Balan et al. 1994). However, as is shown herein, this is not relevant to Δg(GJDI)-2 as an attenuated viral vaccine vector.

Previous literature has indicated that trying to create an HSV recombinant strain by homologous recombination via co-transfection of plasmid DNA and HSV genomic DNA is not efficient; however, literature is scarce on precise estimates of recombination efficiency. Krisky et al. found that using homologous recombination to delete specific nonessential HSV genes resulted in a recombination frequency between 0.05% and 3% (Krisky et al. 1997, Gene Therapy 4: 1120-25). These results showed that the large variety in recombination frequency was due to the effect on the fitness of the recombinant without that gene (Krisky et al. 1997). The Bxb1 mycobacteriophage integrase-mediated recombination was demonstrated herein to be a highly efficient system for constructing recombinant HSV-2 strains. Although the integration of transgenes into the viral genome approaches an efficiency of 100% for inserts up to 6 kb, the stability of the integrated transgene cassette decreases with the increase in size. The high recombination efficiency is clearly an advantage over homologous recombination and represents a much more reliable method of introducing transgenes into an HSV vector. Importantly, the Δg(GJDI)-2 vector expressing a DENV NS1 gene generated a significant antibody response with IgG2b and IgG2c subtypes. DENV in particular is critical as a vaccine candidate. IgG2b and IgG2c isotypes are consistent with high levels of FcγRIV activation and ADCC-mediated protection. It is expected that Δg(GJDI)-2::NS1 will be protective in mice and will demonstrate reduced antibody-dependent enhancement (ADE) activity, a potentially fatal immune reaction when infected a subsequent time with a different DENV serotype, leading to dengue hemorrhagic fever and dengue shock syndrome.

Despite the deletion of four genes, the Δg(GJDI)-2 virus is shown as immunogenic and protective as a strain containing only one gene deletion. The deletion of these genes creates an HSV strain with an additional 6-kB of cloning capacity. The recombination efficiency to 100% by incorporating the attP-attB Bxb1 mycobacteriophage integration system into the Δg(GJDI)-2 vector. Plasmids recombined into this vector remain stable over multiple passages. A novel vaccine construct was generated targeting the DENV2 NS1 protein that is immunogenic with high levels of IgG2b and IgG2c antibodies. The Δg(GJDI)-2 strain and Bxb1 recombination system combine to generate a high-capacity and high-efficiency vector for cloning antigens and other genes into HSV.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise.

Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through carbon of the carbonyl group.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A genetically modified herpes simplex virus (HSV) comprising: a genome comprising a complete deletion of a glycoprotein G-encoding gene, a glycoprotein J-encoding gene, a glycoprotein D-encoding gene, and a glycoprotein I-encoding gene.

2. The genetically modified HSV of claim 1, wherein the genetically modified HSV is herpes simplex virus-1 (HSV-1) or herpes simplex virus-2 (HSV-2).

3. A herpes simplex virus vaccine comprising the genetically modified HSV-2 of claim 1, wherein the HSV-2 is Δ(GJDI)-2.

4. The vaccine of claim 3, further comprising an immunological adjuvant.

5. A method of vaccinating or eliciting an immune response to a HSV-1 or HSV-2 infection or to a co-infection with HSV-1 and HSV-2 in a subject comprising administering the HSV-2 vaccine of claim 3 in an amount effective to vaccinate or elicit the immune response to HSV-1 and/or HSV-2 in the subject.

6. (canceled)

7. A method of eliciting an immune response in a first subject against an HSV-2 and/or HSV-1 infection, comprising effectuating passive transfer to the first subject of an amount of a product from a second subject immunized with the HSV vaccine of claim 3 in an amount effective to elicit the immune response to the HSV-1, HSV-2 in the subject and wherein the product comprises antibodies induced thereby.

8. The method of claim 7, wherein the product comprises serum.

9. The method of claim 7, wherein the first subject and second subject are human.

10. The method of any of claim 7, wherein the first subject is a fetus or neonate.

11. The method of claim 10, wherein the product comprises serum or breast milk of a pregnant female.

12. (canceled)

13. A method for treating an infection with HSV-1 or HSV-2, or a coinfection with HSV-1 and HSV-2, comprising administering the HSV vaccine of claim 3 in an amount effective to treat an HSV-1 or HSV-2 or a coinfection with HSV-1 and HSV-2 in the subject.

14. A product comprising HSV-specific antibodies from a pregnant female immunized with HSV-2 having a complete deletion of genes encoding glycoprotein G, glycoprotein J, glycoprotein D, and glycoprotein I in the genome thereof, wherein said HSV-2 is phenotypically complemented with a herpes simplex virus-1 (HSV-1) glycoprotein G, glycoprotein J, glycoprotein D, and glycoprotein I by propagating said HSV-2 in a complementing cell expressing said HSV-2 glycoprotein G, glycoprotein J, glycoprotein D, and glycoprotein I, for use in eliciting an immune response against an HSV-2 and/or HSV-1 infection in a neonate, wherein the product comprises breast milk of the pregnant female.

15. (canceled)

16. The product of claim 14, wherein the pregnant female is pregnant with a fetus which will become the neonate.

17. The product of claim 14, wherein the pregnant female and the neonate are human.

18. (canceled)

19. (canceled)

20. (canceled)

21. (canceled)

22. The product of claim 14, wherein the complementing cell is a VD60 cell.

23. The product of claim 14, wherein the pregnant female, prior to immunization, is seronegative for HSV-1, seronegative for HSV-2, or seronegative for both HSV-1 and HSV-2.

24. (canceled)

25. (canceled)

26. A genetically modified herpes simplex virus (HSV) gene expression vector for expressing one or more heterologous antigen, comprising:
an HSV-2 or HSV-1 genome comprising a complete deletion of glycoprotein G-encoding gene, glycoprotein J-encoding gene, glycoprotein D-encoding gene, and glycoprotein I-encoding gene;
a heterologous nucleic acid comprising an expression cassette and inserted in a region of the genome from which the glycoprotein G-encoding gene, glycoprotein J-encoding gene, glycoprotein D-encoding gene, and glycoprotein I-encoding gene have been deleted;
an attL sequence; and
an attR sequence,
wherein the attL sequence is adjacent to a first end of the expression cassette and the attR sequence is adjacent to a second end of the expression cassette, and
wherein the expression cassette comprises in operable communication a promoter, one or more gene encoding the one or more heterologous antigen, and optionally, a selectable marker.

27. (canceled)

28. (canceled)

29. (canceled)

30. The HSV gene expression vector of claim 26, wherein the attL sequence has a sequence of SEQ ID NO: 7 and the attR sequence has a sequence of SEQ ID NO: 8.

31. (canceled)

32. (canceled)

33. A method for expressing one or more heterologous protein in a host cell, the method comprising:
contacting a host cell with the HSV gene expression vector of claim 26; and
measuring expression of the heterologous protein in the host cell.

34. A method of producing a herpes simplex virus (HSV) gene expression vector comprising a gene encoding a heterologous protein, the method comprising:
providing a genetically modified HSV-1 or HSV-2 genome comprising
a complete deletion of glycoprotein G-encoding gene, glycoprotein J-encoding gene, glycoprotein D-encoding gene, and glycoprotein I-encoding gene, and
a first recombination sequence inserted in a region of the genome from which the glycoprotein G-encoding gene, glycoprotein J-encoding gene, glycoprotein D-encoding gene, and glycoprotein I-encoding gene have been deleted;
providing a heterologous nucleic acid comprising a second recombination sequence and an expression cassette comprising the gene encoding the heterologous protein, wherein the heterologous protein is chosen from dengue virus serotype 2 NS1 antigen, M. tuberculosis gene DnaK, M. tuberculosis groEL-2, M. tuberculosis cut4, M. tuberculosis cut7, M. tuberculosis 1ppx, and M. tuberculosis pstS1;
an attL sequence; and
an attR sequence,
wherein the attL sequence is adjacent to a first end of the expression cassette and the attR sequence is adjacent to a second end of the expression cassette, and
wherein the expression cassette comprises in operable communication a promoter and at least one gene encoding a heterologous protein;
contacting the genome and the heterologous nucleic acid in the presence of a bacteriophage Bxb1 serine integrase under conditions in which sequence-specific recombination occurs between the first recombination sequence and the second recombination sequence to provide a recombined HSV genome; and
transfecting a complementing cell with the recombined HSV genome to obtain the HSV gene expression vector,
wherein the first recombination sequence comprises a Bxb1 attB sequence from Mycobacterium smegmatis identified in SEQ ID NO: 1 and the second recombination sequence comprises an attP sequence identified in SEQ ID NO: 2 from bacteriophage Bxb1.

35. (canceled)

36. (canceled)

37. (canceled)

38. (canceled)

39. (canceled)

40. (canceled)

41. (canceled)

42. The method of claim 34, wherein the complementing cell expresses HSV-1 glycoprotein G, glycoprotein J, glycoprotein D and glycoprotein I, and phenotypically complements the genetically modified HSV gene expression vector.

43. (canceled)

44. (canceled)

45. (canceled)

46. (canceled)

47. A method of vaccinating or eliciting an immune response to a dengue virus serotype 2 NS1 antigen in a subject comprising administering the HSV vector of claim 34, wherein the heterologous antigen or protein is a dengue virus serotype 2 NS1 antigen, in an amount effective to vaccinate or elicit the immune response to the dengue virus serotype 2 NS1 antigen in the subject.

48. (canceled)

49. A method of eliciting an immune response in a first subject against an HSV-2 and/or HSV-1 infection, and a dengue virus serotype 2 NS1 antigen, comprising effectuating passive transfer to the first subject of an amount of a product from a second subject immunized with the HSV vector of claim 34 wherein the heterologous antigen or protein is a dengue virus serotype 2 NS1 antigen, in an amount effective to elicit the immune response to the HSV-1, HSV-2 and the dengue virus serotype 2 NS1 in the subject and wherein the product comprises antibodies induced thereby.

50. The method of claim 49, wherein the product comprises serum.

51. The method of claim 49, wherein the first subject and second subject are human.

52. The method of claim 49, wherein the first subject is a fetus or neonate.

53. The method of claim 52, wherein the product comprises serum or breast milk of the pregnant female.

54. (canceled)

55. (canceled)

56. A method of vaccinating or eliciting an immune response in a subject against HSV-1, HSV-2, and another pathogen, wherein the pathogen is Mycobacteria tuberculosis, comprising administering the HSV vector of claim 34, wherein the heterologous antigen is any one of M. tuberculosis gene DnaK, M. tuberculosis groEL-2, M. tuberculosis cut4, M. tuberculosis cut7, M. tuberculosis 1ppx, and M. tuberculosis pstS1, in an amount effective to vaccinate or elicit an immune response in the subject against HSV-1, HSV-2, and the Mycobacteria tuberculosis.

57. (canceled)

58. (canceled)

59. A method of eliciting an immune response in a first subject against an HSV-2 and/or HSV-1 infection, and a Mycobacteria tuberculosis antigen, comprising effectuating passive transfer to the first subject of an amount of a product from a second subject immunized with the HSV vector of claim 34, wherein the heterologous antigen is any one of M. tuberculosis gene DnaK, M. tuberculosis groEL-2, M. tuberculosis cut4, M. tuberculosis cut7, M. tuberculosis 1ppx, and M. tuberculosis pstS1, in an amount effective to elicit the immune response to the HSV-1, HSV-2 and the Mycobacteria tuberculosis antigen in the subject and wherein the product comprises antibodies induced thereby.


SEQUENCE LISTING (61 sequences)

SEQ ID NO: 1 - DNA, 38 bp, Mycobacterium smegmatis
  source 1..38: mol_type = genomic DNA; note = attB site
ggcttgtcgacgacggcggtctccgtcgtcaggatcat

SEQ ID NO: 2 - DNA, 48 bp, Synthetic construct
  source 1..48: mol_type = other DNA; note = mycobacteriophage Bxb1 attP site
ggtttgtctggtcaaccaccgcggtctcagtggtgtacggtacaaacc

SEQ ID NO: 3 - protein, 698 aa, Human alphaherpesvirus 2
  source 1..698: note = HSV-2 glycoprotein G
MHAIAPRLLLLFVLSGLPGTRGGSGVPGPINPPNNDVVFPGGSPVAQYCYAYPRLDDPGPLGSADAGRQDLPRRVVRHEPLGRSFLTGGLVLLAPPVRGFGAPNATYAARVTYYRLTRACRQPILLRQYGGCRGGEPPSPKTCGSYTYTYQGGGPPTRYALVNASLLVPIWDRAAETFEYQIELGGELHVGLLWVEVGGEGPGLTAPPQAARAEGGPCVPPVPAGRPWRSVPPVWYSAPNPGFRGLRFRERCLPPQTPAAPSDLPRVAFAPQSLLVGITGRTFIRMARPTEDVGVLPPHWAPGALDDGPYAPFPPRPRFRRALRTDPEGVDPDVRAPRTGRRLMALTENASSDSPTSAPEKTPLPVSATAMAPSVDPSAEPTAPATTTPPDEMATQAATVAVTPEETAVASPPATASVESSPLPAAAATPGAGHTNTSSASAAKTPPTTPAPTTPPPTSTHATPRPTTPGPQTTPPGPATPGPVGASAAPTADSPLTASPPATAPGPSAANVSVAATTATPGTRGTARTPPTDPKTHPHGPADAPPGSPAPPPPEHRGGPEEFEGAGDGEPPEDDDSATGLAFRTPNPNKPPPARPGPIRPTLPPGILGPLAPNTPRPPAQAPAKDMPSGPTPQHIPLFWFLTASPALDILFIISTTIHTAAFVCLVALAAQLWRGRAGRRRYAHPSVRYVCLPPERD

SEQ ID NO: 4 - protein, 92 aa, Human alphaherpesvirus 2
  source 1..92: note = HSV-2 glycoprotein J
MDRYAVRTWGIVGILGCAAVGAAPTGPASDTTNATARLPTHPPLIRSGGFAVPLIVGGLCLMILGMACLLEVLRRLGRELARCCPHAGQFAP

SEQ ID NO: 5 - protein, 393 aa, Human alphaherpesvirus 2
  source 1..393: note = HSV-2 glycoprotein D
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 6 - protein, 372 aa, Human alphaherpesvirus 2
  source 1..372: note = HSV-2 glycoprotein I
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGESAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 7 - DNA, 43 bp, synthetic construct
  source 1..43: mol_type = other DNA; note = attL site
ggcttgtcgacgacggcggtctcagtggtgtacggtacaaacc

SEQ ID NO: 8 - DNA, 43 bp, synthetic construct
  source 1..43: mol_type = other DNA; note = attR site
ggtttgtctggtcaaccaccgcggtctccgtcgtcaggatcat

SEQ ID NO: 9 - DNA, 81 bp, synthetic construct
  source 1..81: mol_type = other DNA; note = gG Forward primer
agacgcggccctcgggctttggtgtttttggcaccttgccgcccggcgtcatttaaatcccaaggacactgagtcctaaag

SEQ ID NO: 10 - DNA, 83 bp, synthetic construct
  source 1..83: mol_type = other DNA; note = gI reverse primer
gggggggaaataaccacgatgggggcggtggggcgggcctgccgaacggccatttaaataagaaggtgttgctgactcatacc

SEQ ID NO: 11 - DNA, 34 bp, synthetic construct
  source 1..34: mol_type = other DNA; note = Primer G194_2904_attB-F
gcgcgcgcttataactcgagccggcttgtcgacg

SEQ ID NO: 12 - DNA, 34 bp, synthetic construct
  source 1..34: mol_type = other DNA; note = Primer G194_2944_attBR
gcgcgcgcttataaatgatcctgacgacggagac

SEQ ID NO: 13 - protein, 21 aa, synthetic construct
  source 1..21: note = 1..3 added to thosea asigna virus 2A sequence
GSGEGRGSLLTCGDVEENPGP

SEQ ID NO: 14 - protein, 22 aa, synthetic construct
  source 1..22: note = 1..3 added to porcine teschovirus-1 2A protein
GSGATNFSLLKQAGDVEENPGP

SEQ ID NO: 15 - protein, 23 aa, synthetic construct
  source 1..23: note = 1..3 added to equine rhinitis A virus
GSGQCTNYALLKLAGDVESNPGP

SEQ ID NO: 16 - protein, 25 aa, synthetic construct
  source 1..25: note = 1..3 added to foot-and-mouth disease virus 2A protein
GSGVKQTLNFDLLKLAGDVESNPGP

SEQ ID NO: 17 - DNA, 20 bp, synthetic construct
  source 1..20: mol_type = other DNA; note = US3 FW primer
actaccctcatcgggtaatc

SEQ ID NO: 18 - DNA, 20 bp, synthetic construct
  source 1..20: mol_type = other DNA; note = US3 RV primer
aggttacccgtttccaggac

SEQ ID NO: 19 - DNA, 55 bp, synthetic construct
  source 1..55: mol_type = other DNA; note = Amp D2gen Mv5pD F primer
actgctagacctgcaggtcagtggtggtggtggtggtgttcctcacgtggcttct

SEQ ID NO: 20 - DNA, 55 bp, synthetic construct
  source 1..55: mol_type = other DNA; note = Amp D2gen pv5pD R primer
actgctagagtttaaactcagtggtggtggtggtggtgttcctcacgtggcttct

SEQ ID NO: 21 - DNA, 31 bp, synthetic construct
  source 1..31: mol_type = other DNA; note = HFVecp91 Hsp70 R primer
gaccgcacgagccatgtcaccagtggaacct

SEQ ID NO: 22 - DNA, 31 bp, synthetic construct
  source 1..31: mol_type = other DNA; note = Primer HFVecp91_Hsp70_F
acggccgggaggccaaggctgtgggccagga

SEQ ID NO: 23 - DNA, 31 bp, synthetic construct
  source 1..31: mol_type = other DNA; note = Primer HFInsHsp70_p91_F
aggttccactggtgacatggctcgtgcggtc

SEQ ID NO: 24 - DNA, 33 bp, synthetic construct
  source 1..33: mol_type = other DNA; note = Primer HFInsHsp70_p91_R
cgtgtcctggcccacagccttggcctcccggcc

SEQ ID NO: 25 - DNA, 41 bp, synthetic construct
  source 1..41: mol_type = other DNA; note = Primer HFInspHsp65_p97_F
ttccaggttccactggtgacatggccaagacaattgcgtac

SEQ ID NO: 26 - DNA, 38 bp, synthetic construct
  source 1..38: mol_type = other DNA; note = Primer HFInspHsp65_p97_R
gtgtcctggcccacagcgaaatccatgccacccatgtc

SEQ ID NO: 27 - DNA, 41 bp, synthetic construct
  source 1..41: mol_type = other DNA; note = Primer HFVecpHsp65_p97_F
gtacgcaattgtcttggccatgtcaccagtggaacctggaa

SEQ ID NO: 28 - DNA, 38 bp, synthetic construct
  source 1..38: mol_type = other DNA; note = Primer HFVecpHsp65_p97_R
gacatgggtggcatggatttcgctgtgggccaggacac

SEQ ID NO: 29 - DNA, 977 bp, synthetic construct
  source 1..977: mol_type = other DNA; note = DENV2 NS1 codon optimized
gtgcggcagcggcatcttcatcaccgacaatgtgcacacctggaccgagcagtacaagttccagcctgagagcccttctaagctggcctctgccatccagaaggcccaagaagagggcatctgcggcatcagaagcgtgaccagactggaaaacctgatgtggaagcagatcacccctgagctgaaccacatcctggccgagaacgaagtgaagctgaccatcatgaccggcgacatcaagggcatcatgcaggccggaaaaagaagcctgaggccacagcctaccgagctgaagtacagctggaaaacatggggcaaagccaagatgctgagcaccgagagccacaaccagacctttctgatcgacggccctgagacagccgagtgtcccaataccaacagagcctggaacagcctggaagtggaagattacggcttcggcgtgttcaccaccaacatctggctgaagctgaaagagaagcaggacgccttctgcgacagcaagctgatgtctgccgccatcaaggacaatagagccgtgcacgccgacatgggctattggatcgagagcgccctgaacgacacctggaagatcgagaaggcctccttcatcgaagtcaagaactgccactggcctaagagccacacactgtggtccaatggcgtgctggaaagcgagatgatcattcccaagaacctggctggccccgtgtctcagcacaactacagacctggctaccacacacagatcgccggaccttggcacctgggcaagctggaaatggacttcgatttctgcgacggcaccaccgtggtggtcaccgaggattgtggcaatagaggccctagcctgagaaccacaacagccagcggaaagctgatcaccgagtgctgctgcagaagctgtaccctgcctcctctgaaatacagaagcgaggatggctggtggtaccgcatggaaatcaggcccctcagag

SEQ ID NO: 30 - DNA, 1878 bp, Mycobacterium tuberculosis
  source 1..1878: mol_type = genomic DNA; note = locus Rv0350, dnaK
atggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcagtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggcggcgagccgggcggtgcccaccccggctcggctgatgacgttgtggacgcggaggtggtcgacgacggccgggaggccaagtga

SEQ ID NO: 31 - protein, 625 aa, Mycobacterium tuberculosis
  source 1..625: note = Mycobacterium tuberculosis H37Rv|Rv0350|dnaK
MARAVGIDLGTTNSVVSVLEGGDPVVVANSEGSRTTPSIVAFARNGEVLVGQPAKNQAVTNVDRTVRSVKRHMGSDWSIEIDGKKYTAPEISARILMKLKRDAEAYLGEDITDAVITTPAYFNDAQRQATKDAGQIAGLNVLRIVNEPTAAALAYGLDKGEKEQRILVFDLGGGTFDVSLLEIGEGVVEVRATSGDNHLGGDDWDQRVVDWLVDKFKGTSGIDLTKDKMAMQRLREAAEKAKIELSSSQSTSINLPYITVDADKNPLFLDEQLTRAEFQRITQDLLDRTRKPFQSVIADTGISVSEIDHVVLVGGSTRMPAVTDLVKELTGGKEPNKGVNPDEVVAVGAALQAGVLKGEVKDVLLLDVTPLSLGIETKGGVMTRLIERNTTIPTKRSETFTTADDNQPSVQIQVYQGEREIAAHNKLLGSFELTGIPPAPRGIPQIEVTFDIDANGIVHVTAKDKGTGKENTIRIQEGSGLSKEDIDRMIKDAEAHAEEDRKRREEADVRNQAETLVYQTEKFVKEQREAEGGSKVPEDTLNKVDAAVAEAKAALGGSDISAIKSAMEKLGQESQALGQAIYEAAQAASQATGAAHPGGEPGGAHPGSADDVVDAEVVDDGREAK

SEQ ID NO: 32 - DNA, 1623 bp, Mycobacterium tuberculosis
  source 1..1623: mol_type = genomic DNA; note = Mycobacterium tuberculosis H37Rv|CDS|Rv0440|528608-530230|+|groEL2|
atggccaagacaattgcgtacgacgaagaggcccgtcgcggcctcgagcggggcttgaacgccctcgccgatgcggtaaaggtgacattgggccccaagggccgcaacgtcgtcctggaaaagaagtggggtgcccccacgatcaccaacgatggtgtgtccatcgccaaggagatcgagctggaggatccgtacgagaagatcggcgccgagctggtcaaagaggtagccaagaagaccgatgacgtcgccggtgacggcaccacgacggccaccgtgctggcccaggcgttggttcgcgagggcctgcgcaacgtcgcggccggcgccaacccgctcggtctcaaacgcggcatcgaaaaggccgtggagaaggtcaccgagaccctgctcaagggcgccaaggaggtcgagaccaaggagcagattgcggccaccgcagcgatttcggcgggtgaccagtccatcggtgacctgatcgccgaggcgatggacaaggtgggcaacgagggcgtcatcaccgtcgaggagtccaacacctttgggctgcagctcgagctcaccgagggtatgcggttcgacaagggctacatctcggggtacttcgtgaccgacccggagcgtcaggaggcggtcctggaggacccctacatcctgctggtcagctccaaggtgtccactgtcaaggatctgctgccgctgctcgagaaggtcatcggagccggtaagccgctgctgatcatcgccgaggacgtcgagggcgaggcgctgtccaccctggtcgtcaacaagatccgcggcaccttcaagtcggtggcggtcaaggctcccggcttcggcgaccgccgcaaggcgatgctgcaggatatggccattctcaccggtggtcaggtgatcagcgaagaggtcggcctgacgctggagaacgccgacctgtcgctgctaggcaaggcccgcaaggtcgtggtcaccaaggacgagaccaccatcgtcgagggcgccggtgacaccgacgccatcgccggacgagtggcccagatccgccaggagatcgagaacagcgactccgactacgaccgtgagaagctgcaggagcggctggccaagctggccggtggtgtcgcggtgatcaaggccggtgccgccaccgaggtcgaactcaaggagcgcaagcaccgcatcgaggatgcggttcgcaatgccaaggccgccgtcgaggagggcatcgtcgccggtgggggtgtgacgctgttgcaagcggccccgaccctggacgagctgaagctcgaaggcgacgaggcgaccggcgccaacatcgtgaaggtggcgctggaggccccgctgaagcagatcgccttcaactccgggctggagccgggcgtggtggccgagaaggtgcgcaacctgccggctggccacggactgaacgctcagaccggtgtctacgaggatctgctcgctgccggcgttgctgacccggtcaaggtgacccgttcggcgctgcagaatgcggcgtccatcgcggggctgttcctgaccaccgaggccgtcgttgccgacaagccggaaaaggagaaggcttccgttcccggtggcggcgacatgggtggcatggatttctga

SEQ ID NO: 33 - protein, 540 aa, Mycobacterium tuberculosis
  source 1..540: note = Mycobacterium tuberculosis H37Rv|Rv0440|groEL2
MAKTIAYDEEARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEKVTETLLKGAKEVETKEQIAATAAISAGDQSIGDLIAEAMDKVGNEGVITVEESNTFGLQLELTEGMRFDKGYISGYFVTDPERQEAVLEDPYILLVSSKVSTVKDLLPLLEKVIGAGKPLLIIAEDVEGEALSTLVVNKIRGTFKSVAVKAPGFGDRRKAMLQDMAILTGGQVISEEVGLTLENADLSLLGKARKVVVTKDETTIVEGAGDTDAIAGRVAQIRQEIENSDSDYDREKLQERLAKLAGGVAVIKAGAATEVELKERKHRIEDAVRNAKAAVEEGIVAGGGVTLLQAAPTLDELKLEGDEATGANIVKVALEAPLKQIAFNSGLEPGVVAEKVRNLPAGHGLNAQTGVYEDLLAAGVADPVKVTRSALQNAASIAGLFLTTEAVVADKPEKEKASVPGGGDMGGMDF

SEQ ID NO: 34 - DNA, 147 bp, Homo sapiens
  source 1..147: mol_type = genomic DNA; note = PDGFR-TM: human platelet-derived growth factor receptor transmembrane domain
gctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 35 - DNA, 63 bp, Mus musculus
  source 1..63: mol_type = genomic DNA; note = mus musculus immunoglobulin kappa leader signal sequence; IgK
atggagacagacacactcctgctatgggtactgctgctctgggttccaggttccactggtgac

SEQ ID NO: 36 - DNA, 166 bp, Human alphaherpesvirus 1
  source 1..166: mol_type = genomic DNA; note = Herpes simplex virus serotype 1 glycoprotein D promoter
atcccctaagggggaggggccattttacgaggaggaggggtataacaaagtctgtctttaaaaagcaggggttagggagttgttcggtcataagcttcagcgcgaacgaccaactaccccgatcatcagttatccttaaggtctcttttgtgtggtgcgttccggt

SEQ ID NO: 37 - DNA, 169 bp, synthetic construct
  source 1..169: mol_type = other DNA; note = modified HSV-1 minimal promoter
cgaatcccctaagggggaggggccattttacgaggaggaggggtataacaaagtctgtctttaaaaagcaggggttagggagttgttcggtcataagcttcagcgcgaacgaccaactaccccgatcatcagttatccttaaggtctcttttgtgtggtgcgttccggt

SEQ ID NO: 38 - DNA, 654 bp, Mycobacterium tuberculosis
  source 1..654: mol_type = genomic DNA; note = Mycobacterium tuberculosis H37Rv|CDS|Rv1984c|2227908-2228561|-|cfp21
atgactccacgcagccttgttcgcatcgttggtgtcgtggttgcgacgaccttggcgctggtgagcgcacccgccggcggtcgtgccgcgcatgcggatccgtgttcggacatcgcggtcgttttcgctcgcggcacgcatcaggcttctggtcttggcgacgtcggtgaggcgttcgtcgactcgcttacctcgcaagttggcgggcggtcgattggggtctacgcggtgaactacccagcaagcgacgactaccgcgcgagcgcgtcaaacggttccgatgatgcgagcgcccacatccagcgcaccgtcgccagctgcccgaacaccaggattgtgcttggtggctattcgcagggtgcgacggtcatcgatttgtccacctcggcgatgccgcccgcggtggcagatcatgtcgccgctgtcgcccttttcggcgagccatccagtggtttctccagcatgttgtggggcggcgggtcgttgccgacaatcggtccgctgtatagctctaagaccataaacttgtgtgctcccgacgatccaatatgcaccggaggcggcaatattatggcgcatgtttcgtatgttcagtcggggatgacaagccaggcggcgacattcgcggcgaacaggctcgatcacgccggatga

SEQ ID NO: 39 - protein, 217 aa, Mycobacterium tuberculosis
  source 1..217: note = Mycobacterium tuberculosis H37Rv|Rv1984c|cfp21
MTPRSLVRIVGVVVATTLALVSAPAGGRAAHADPCSDIAVVFARGTHQASGLGDVGEAFVDSLTSQVGGRSIGVYAVNYPASDDYRASASNGSDDASAHIQRTVASCPNTRIVLGGYSQGATVIDLSTSAMPPAVADHVAAVALFGEPSSGFSSMLWGGGSLPTIGPLYSSKTINLCAPDDPICTGGGNIMAHVSYVQSGMTSQAATFAANRLDHAG

SEQ ID NO: 40 - DNA, 681 bp, Mycobacterium tuberculosis
  source 1..681: mol_type = genomic DNA; note = Mycobacterium tuberculosis H37Rv|CDS|Rv3452|3873452-3874132|+|cut4
gtgatcccacgcccgcagccgcattcgggccgctggcgagccggtgccgcacgccgcctcaccagcctggtggccgccgcctttgcggcggccacactgttgcttacccccgcgctggcaccaccggcatcggcgggctgcccggatgccgaggtggtgttcgcccgcggaaccggcgaaccacctggcctcggtcgggtaggccaagctttcgtcagttcattgcgccagcagaccaacaagagcatcgggacatacggagtcaactacccggccaacggtgatttcttggccgccgctgacggcgcgaacgacgccagcgaccacattcagcagatggccagcgcgtgccgggccacgaggttggtgctcggcggctactcccagggtgcggccgtgatcgacatcgtcaccgccgcaccactgcccggcctcgggttcacgcagccgttgccgcccgcagcggacgatcacatcgccgcgatcgccctgttcgggaatccctcgggccgcgctggcgggctgatgagcgccctgacccctcaattcgggtccaagaccatcaacctctgcaacaacggcgacccgatttgttcggacggcaaccggtggcgagcgcacctaggctacgtgcccgggatgaccaaccaggcggcgcgtttcgtcgcgagcaggatctaa

SEQ ID NO: 41 - protein, 226 aa, Mycobacterium tuberculosis
  source 1..226: note = Mycobacterium tuberculosis H37Rv|Rv3452|cut4
VIPRPQPHSGRWRAGAARRLTSLVAAAFAAATLLLTPALAPPASAGCPDAEVVFARGTGEPPGLGRVGQAFVSSLRQQTNKSIGTYGVNYPANGDFLAAADGANDASDHIQQMASACRATRLVLGGYSQGAAVIDIVTAAPLPGLGFTQPLPPAADDHIAAIALFGNPSGRAGGLMSALTPQFGSKTINLCNNGDPICSDGNRWRAHLGYVPGMTNQAARFVASRI

SEQ ID NO: 42 - DNA, 702 bp, Mycobacterium tuberculosis
  source 1..702: mol_type = genomic DNA; note = Mycobacterium tuberculosis H37Rv|CDS|Rv2945c|3290624-3291325|-|lppX
atgaatgatggaaaacgggcggtgacgtccgctgttctggtggtgctaggtgcctgcctggcgttgtggctatcaggatgttcttcgccgaaacctgatgccgaggaacagggtgttcccgtgagcccgacggcgtccgaccccgcgctcctcgccgagatcaggcagtcgcttgatgcgacaaaagggttgaccagcgtgcacgtagcggtccgaacaaccgggaaagtcgacagcttgctgggtattaccagtgccgatgtcgacgtccgggccaatccgctcgcggcaaagggcgtatgcacctacaacgacgagcagggtgtcccgtttcgggtacaaggcgacaacatctcggtgaaactgttcgacgactggagcaatctcggctcgatttctgaactgtcaacttcacgcgtgctcgatcctgccgctggggtgacgcagctgctgtccggtgtcacgaacctccaagcgcaaggtaccgaagtgatagacggaatttcgaccaccaaaatcaccgggaccatccccgcgagctctgtcaagatgcttgatcctggcgccaagagtgcaaggccggcgaccgtgtggattgcccaggacggctcgcaccacctcgtccgagcgagcatcgacctcggatccgggtcgattcagctcacgcagtcgaaatggaacgaacccgtcaacgtcgactag

SEQ ID NO: 43 - protein, 233 aa, Mycobacterium tuberculosis
  source 1..233: note = Mycobacterium tuberculosis H37Rv|Rv2945c|lppX
MNDGKRAVTSAVLVVLGACLALWLSGCSSPKPDAEEQGVPVSPTASDPALLAEIRQSLDATKGLTSVHVAVRTTGKVDSLLGITSADVDVRANPLAAKGVCTYNDEQGVPFRVQGDNISVKLFDDWSNLGSISELSTSRVLDPAAGVTQLLSGVTNLQAQGTEVIDGISTTKITGTIPASSVKMLDPGAKSARPATVWIAQDGSHHLVRASIDLGSGSIQLTQSKWNEPVNVD

SEQ ID NO: 44 - DNA, 1125 bp, Mycobacterium tuberculosis
  source 1..1125: mol_type = genomic DNA; note = Mycobacterium tuberculosis H37Rv|CDS|Rv0934|1042115-1043239|+|pstS1
gtgaaaattcgtttgcatacgctgttggccgtgttgaccgctgcgccgctgctgctagcagcggcgggctgtggctcgaaaccaccgagcggttcgcctgaaacgggcgccggcgccggtactgtcgcgactacccccgcgtcgtcgccggtgacgttggcggagaccggtagcacgctgctctacccgctgttcaacctgtggggtccggcctttcacgagaggtatccgaacgtcacgatcaccgctcagggcaccggttctggtgccgggatcgcgcaggccgccgccgggacggtcaacattggggcctccgacgcctatctgtcggaaggtgatatggccgcgcacaaggggctgatgaacatcgcgctagccatctccgctcagcaggtcaactacaacctgcccggagtgagcgagcacctcaagctgaacggaaaagtcctggcggccatgtaccagggcaccatcaaaacctgggacgacccgcagatcgctgcgctcaaccccggcgtgaacctgcccggcaccgcggtagttccgctgcaccgctccgacgggtccggtgacaccttcttgttcacccagtacctgtccaagcaagatcccgagggctggggcaagtcgcccggcttcggcaccaccgtcgacttcccggcggtgccgggtgcgctgggtgagaacggcaacggcggcatggtgaccggttgcgccgagacaccgggctgcgtggcctatatcggcatcagcttcctcgaccaggccagtcaacggggactcggcgaggcccaactaggcaatagctctggcaatttcttgttgcccgacgcgcaaagcattcaggccgcggcggctggcttcgcatcgaaaaccccggcgaaccaggcgatttcgatgatcgacgggcccgccccggacggctacccgatcatcaactacgagtacgccatcgtcaacaaccggcaaaaggacgccgccaccgcgcagaccttgcaggcatttctgcactgggcgatcaccgacggcaacaaggcctcgttcctcgaccaggttcatttccagccgctgccgcccgcggtggtgaagttgtctgacgcgttgatcgcgacgatttccagctag

SEQ ID NO: 45 - protein, 374 aa, Mycobacterium tuberculosis
  source 1..374: note = Mycobacterium tuberculosis H37Rv|Rv0934|pstS1
VKIRLHTLLAVLTAAPLLLAAAGCGSKPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISS

SEQ ID NO: 46 - DNA, 40 bp, Synthetic construct
  source 1..40: mol_type = other DNA; note = Primer Cut7_New_F
ccatatgatgttccagattatgctatgactccacgcagcc

SEQ ID NO: 47 - DNA, 39 bp, synthetic construct
  source 1..39: mol_type = other DNA; note = Primer Cut7_New_R
gtgtcctggcccacagcatttccggcgtgatcgagcctg

SEQ ID NO: 48 - DNA, 30 bp, Synthetic construct
  source 1..30: mol_type = other DNA; note = Primer pMAE83_Cut7_BB_F
agcataatctggaacatcatatggatagtc

SEQ ID NO: 49 - DNA, 24 bp, synthetic construct
  source 1..24: mol_type = other DNA; note = pMAE83_Cut7_BB_R
aatgctgtgggccaggacacgcag

SEQ ID NO: 50 - DNA, 41 bp, synthetic construct
  source 1..41: mol_type = other DNA; note = Cut4_New_F
tatgatgttccagattatgctgtgatccccgcccgcagccg

SEQ ID NO: 51 - DNA, 40 bp, synthetic construct
  source 1..40: mol_type = other DNA; note = Cut4_New_R
gtcctggcccacagcattgatcctgctcgcgacgaaacgc

SEQ ID NO: 52 - DNA, 24 bp, synthetic construct
  source 1..24: mol_type = other DNA; note = Primer pMAE83_Cut4_BB_F
aatgctgtgggccaggacacgcag

SEQ ID NO: 53 - DNA, 32 bp, synthetic construct
  source 1..32: mol_type = other DNA; note = Primer pMAE83_Cut4_BB_R
agcataatctggaacatcatatggatagtcac

SEQ ID NO: 54 - DNA, 18 bp, synthetic construct
  source 1..18: mol_type = other DNA; note = Primer pMAE97F
gctgtgggccaggacacg

SEQ ID NO: 55 - DNA, 21 bp, synthetic construct
  source 1..21: mol_type = other DNA; note = Primer pMAE97R
gtcaccagtggaacctggaac

SEQ ID NO: 56 - DNA, 45 bp, synthetic construct
  source 1..45: mol_type = other DNA; note = Primer Rv2945cF
ctgggttccaggttccactggtgacatgaatgatggaaaacgggc

SEQ ID NO: 57 - DNA, 43 bp, synthetic construct
  source 1..43: mol_type = other DNA; note = Primer Rv2945cR
cctcctgcgtgtcctggcccacagcgtcgacgttgacgggttc

SEQ ID NO: 58 - DNA, 18 bp, synthetic construct
  source 1..18: mol_type = other DNA; note = Primer pMAE97F
gctgtgggccaggacacg

SEQ ID NO: 59 - DNA, 21 bp, synthetic construct
  source 1..21: mol_type = other DNA; note = Primer pMAE97R
gtcaccagtggaacctggaac

SEQ ID NO: 60 - DNA, 47 bp, synthetic construct
  source 1..47: mol_type = other DNA; note = Primer Rv0934cF
ctgggttccaggttccactggtgacatgaaaattcgtttgcatacgc

SEQ ID NO: 61 - DNA, 43 bp, synthetic construct
  source 1..43: mol_type = other DNA; note = Primer Rv0934cR
cctcctgcgtgtcctggcccacagcgctggaaatcgtcgcgat